(19) United States
(12) Patent Application Publication
(10) Pub. No.: US 2024/0412043 A1
(43) Pub. Date: Dec. 12, 2024

(54) METHOD AND APPARATUS FOR TRAINING NOISE DATA DETERMINING MODEL AND DETERMINING NOISE DATA

(71) Applicant: Tencent Technology (Shenzhen) Company Limited, Shenzhen (CN)

(72) Inventors: Lei HUANG, Shenzhen (CN); Hengtong ZHANG, Shenzhen (CN); Tingyang XU, Shenzhen (CN)

(73) Assignee: Tencent Technology (Shenzhen) Company Limited, Shenzhen, GD (CN)

(21) Appl. No.: 18/805,224

(22) Filed: Aug. 14, 2024

Related U.S. Application Data
(63) Continuation of application No. PCT/CN2023/125347, filed on Oct. 19, 2023.

(30) Foreign Application Priority Data
Nov. 30, 2022 (CN) 202211525333.7

(51) Int. Cl. G06N 3/0455

(52) U.S. Cl. CPC G06N 3/0455

(57) ABSTRACT

This application discloses training a noise data determining model and determining noise data. A method includes: obtaining sample noisy small molecule data and annotated noise data, the sample noisy small molecule data including data of a plurality of sample atoms; outputting a sample graph structure by using a neural network model based on the data of the plurality of sample atoms; performing prediction on the sample graph structure by using the neural network model, to obtain predicted noise data; and training the neural network model based on the predicted noise data and the annotated noise data, to obtain a noise data determining model. Final noise data in to-be-processed noisy small molecule data is determined by using the noise data determining model, so that denoising processing can be performed on the to-be-processed noisy small molecule data based on the final noise data, to obtain denoised small molecule dat

### RELATED APPLICATION

This application is a continuation of International Patent Application No. PCT/CN2023/125347, filed Oct. 19, 2023, which claims priority to Chinese Patent Application No. 202211525333.7, filed with the China National Intellectual Property Administration on Nov. 30, 2022 and entitled “METHOD AND APPARATUS FOR TRAINING NOISE DATA DETERMINING MODEL AND DETERMINING NOISE DATA”. The contents of International Patent Application No. PCT/CN2023/125347 and Chinese Patent Application No. 202211525333.7 are each incorporated herein by reference in their entirety.

### FIELD OF THE TECHNOLOGY

Embodiments of this application relate to the field of biotechnologies, and in particular, to a method and an apparatus for training a noise data determining model and determining noise data.

### BACKGROUND OF THE DISCLOSURE

In the field of biotechnologies, small molecules are related to drug research and development. In some cases, randomly generated noise data may be used as noisy small molecule data, and noise data in the noisy small molecule data is determined, to perform denoising processing on the noisy small molecule data based on the noise data, to obtain denoised small molecule data. If the denoised small molecule data meets a condition, the denoised small molecule data is used as target small molecule data without noise. If the denoised small molecule data does not meet the condition, the denoised small molecule data is used as noisy small molecule data, and denoising processing is performed on the noisy small molecule data again until the condition is met, to obtain the target small molecule data. The target small molecule data can be used to perform drug research and development, and to speed up the drug research and development rate. Based on this, how to determine the noise data in the noisy small molecule data becomes a problem urgently to be resolved.

### SUMMARY

This application provides a method and an apparatus for training a noise data determining model and determining noise data, which can be used to resolve problems in the related art. Technical solutions include the following content.

According to a first aspect, a method for training a noise data determining model is provided, performed by an electronic device. The method includes:
obtaining sample noisy small molecule data and annotated noise data, the sample noisy small molecule data being small molecule data with noise data and the sample noisy small molecule data including data of a plurality of sample atoms, and the annotated noise data being noise data obtained from the sample noisy small molecule data through annotation;
outputting a sample graph structure by using a neural network model based on the data of the plurality of sample atoms, the sample graph structure including a plurality of sample nodes and a plurality of sample edges, any sample node representing data of one sample atom, and any sample edge representing a distance between sample atoms corresponding to two sample nodes at two ends of the sample edge;
performing prediction on the sample graph structure by using the neural network model, to obtain predicted noise data, the predicted noise data being noise data obtained from the sample noisy small molecule data through prediction; and
training the neural network model based on the predicted noise data and the annotated noise data, to obtain a noise data determining model, the noise data determining model being configured to determine final noise data in to-be-processed noisy small molecule data.

According to a second aspect, a method for determining noise data, performed by an electronic device. The method includes:
obtaining to-be-processed noisy small molecule data, the to-be-processed noisy small molecule data being small molecule data with noise data and the to-be-processed noisy small molecule data including data of a plurality of to-be-processed atoms;
determining a to-be-processed graph structure by using a noise data determining model based on the data of the plurality of to-be-processed atoms, the to-be-processed graph structure including a plurality of nodes and a plurality of edges, any node representing one to-be-processed atom, any edge representing a distance between to-be-processed atoms corresponding to two nodes at two ends of the edge, and the noise data determining model being obtained through training according to the method according to any one of descriptions in the first aspect; and
determining final noise data by using the noise data determining model based on the to-be-processed graph structure, the final noise data being noise data in the to-be-processed noisy small molecule data.

According to a third aspect, an apparatus for training a noise data determining model is provided, deployed on an electronic device. The apparatus includes:
an obtaining module, configured to obtain sample noisy small molecule data and annotated noise data, the sample noisy small molecule data being small molecule data with noise data and the sample noisy small molecule data including data of a plurality of sample atoms, and the annotated noise data being noise data obtained from the sample noisy small molecule data through annotation;
a determining module, configured to output a sample graph structure by using a neural network model based on the data of the plurality of sample atoms, the sample graph structure including a plurality of sample nodes and a plurality of sample edges, any sample node representing data of one sample atom, and any sample edge representing a distance between sample atoms corresponding to two sample nodes at two ends of the sample edge,
the determining module being further configured to perform prediction on the sample graph structure by using the neural network model, to obtain predicted noise data, the predicted noise data being noise data obtained from the sample noisy small molecule data through prediction; and
a training module, configured to train the neural network model based on the predicted noise data and the annotated noise data, to obtain a noise data determining model, the noise data determining model being configured to determine final noise data in to-be-processed noisy small molecule data.

According to a fourth aspect, an apparatus for determining noise data is provided, deployed on an electronic device. The apparatus includes:
an obtaining module, configured to obtain to-be-processed noisy small molecule data, the to-be-processed noisy small molecule data being small molecule data with noise data and the to-be-processed noisy small molecule data including data of a plurality of to-be-processed atoms; and
a determining module, configured to determine a to-be-processed graph structure by using a noise data determining model based on the data of the plurality of to-be-processed atoms, the to-be-processed graph structure including a plurality of nodes and a plurality of edges, any node representing one to-be-processed atom, any edge representing a distance between to-be-processed atoms corresponding to two nodes at two ends of the edge, and the noise data determining model being obtained through training according to the method for training a noise data determining model according to any one of descriptions in the first aspect,
the determining module being further configured to determine final noise data by using the noise data determining model based on the to-be-processed graph structure, the final noise data being noise data in the to-be-processed noisy small molecule data.

According to a fifth aspect, an electronic device is provided. The electronic device includes a processor and a memory, the memory having at least one computer program stored therein, the at least one computer program being loaded and executed by the processor, to cause the electronic device to implement the method for training a noise data determining model according to any one of descriptions in the first aspect or implement the method for determining noise data according to any one of descriptions in the second aspect.

According to a sixth aspect, a computer-readable storage medium is further provided. The computer-readable storage medium has at least one computer program stored therein, the at least one computer program being loaded and executed by a processor, to cause an electronic device to implement the method for training a noise data determining model according to any one of descriptions in the first aspect or implement the method for determining noise data according to any one of descriptions in the second aspect.

According to a seventh aspect, a computer program product is further provided. The computer program product has at least one computer program stored therein, the at least one computer program being loaded and executed by a processor, to cause an electronic device to implement the method for training a noise data determining model according to any one of descriptions in the first aspect or implement the method for determining noise data according to any one of descriptions in the second aspect.

The technical solutions provided in embodiments of this application at least bring the following beneficial effects:

In the technical solutions provided in this application, a sample graph structure is determined based on data of a plurality of sample atoms in sample noisy small molecule data, prediction is performed on the sample graph structure by using a neural network model, to determine predicted noise data, and training is performed based on the predicted noise data and annotated noise data, to obtain a noise data determining model. Final noise data in to-be-processed noisy small molecule data may be determined by using the noise data determining model, so that denoising processing can be performed on the to-be-processed noisy small molecule data based on the final noise data, to obtain denoised small molecule data. In this way, drug research and development can be performed based on the denoised small molecule data, thereby improving drug research and development efficiency.

### BRIEF DESCRIPTION OF THE DRAWINGS

To describe the technical solutions in embodiments of this application more clearly, the following briefly describes the accompanying drawings required for describing the embodiments. Apparently, the accompanying drawings in the following descriptions show merely some embodiments of this application, and a person of ordinary skill in the art may derive other drawings from these accompanying drawings without creative efforts.

FIG. 1 is a schematic diagram of an implementation environment of a method for training a noise data determining model or a method for determining noise data according to an embodiment of this application.

FIG. 2 is a flowchart of a method for training a noise data determining model according to an embodiment of this application.

FIG. 3 is a schematic diagram of noise processing and denoising processing according to an embodiment of this application.

FIG. 4 is a flowchart of a method for determining noise data according to an embodiment of this application.

FIG. 5 is a schematic diagram of a process of training a noise data determining model according to an embodiment of this application.

FIG. 6 is a schematic diagram of a target small molecule according to an embodiment of this application.

FIG. 7 is a structural schematic diagram of an apparatus for training a noise data determining model according to an embodiment of this application.

FIG. 8 is a schematic structural diagram of an apparatus for determining noise data according to an embodiment of this application.

FIG. 9 is a schematic structural diagram of a terminal device according to an embodiment of this application.

FIG. 10 is a schematic structural diagram of a server according to an embodiment of this application.

### DESCRIPTION OF EMBODIMENTS

To make the objectives, technical solutions, and advantages of this application clearer, implementations of this application are further described below in detail with reference to the accompanying drawings.

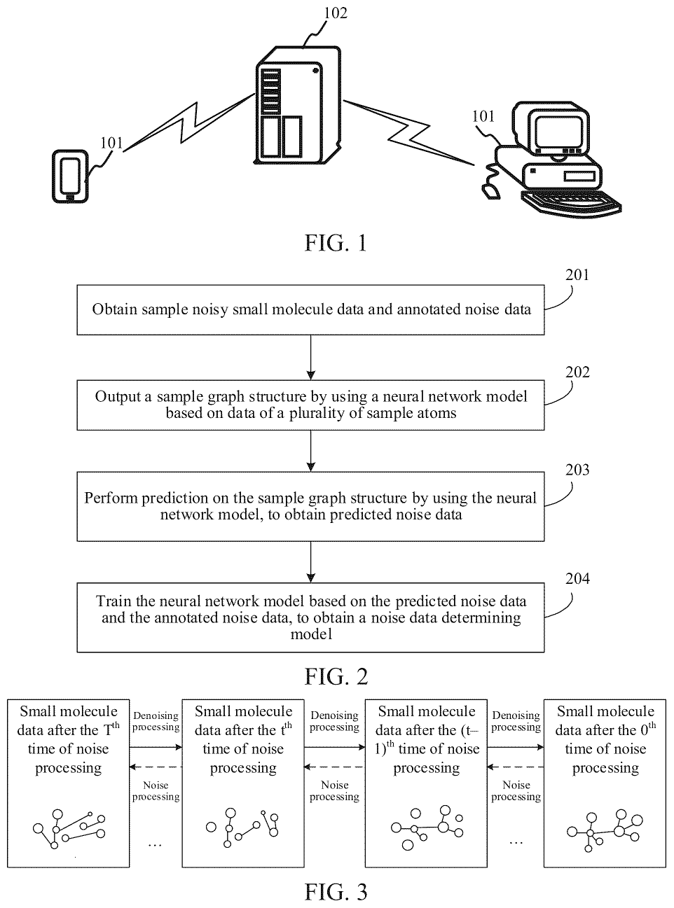

FIG. 1 is a schematic diagram of an implementation environment of a method for training a noise data determining model or a method for determining noise data according to an embodiment of this application. As shown in FIG. 1, the implementation environment includes a terminal device 101 and a server 102. The method for training a noise data determining model or the method for determining noise data provided in the embodiments of this application may be performed by the terminal device 101, or may be performed by the server 102, or may be jointly performed by the terminal device 101 and the server 102.

The terminal device 101 may be a smartphone, a game console, a desktop computer, a tablet computer, a laptop portable computer, a smart television, a smart in-vehicle device, a smart voice interaction device, a smart home appliance, or the like. The server 102 may be at least one of one server, a server cluster including a plurality of servers, a cloud computing platform, or a virtualization center. This is not limited in the embodiments of this application. The server 102 may communicate with the terminal device 101 by using a wired or wireless network. The server 102 may have functions of data processing, data storage, data transmitting and receiving, and the like. This is not limited in the embodiments of this application. A number of terminal devices 101 and a number of servers 102 are not limited, and there may be one or more terminal devices 101 or servers 102.

In the embodiments of this application, the method for training a noise data determining model or the method for determining noise data may be automatically performed based on an artificial intelligence technology.

In the field of biotechnologies, randomly generated noise data may be used as noisy small molecule data, and noise data in the noisy small molecule data is determined, to perform denoising processing on the noisy small molecule data based on the noise data. When denoised small molecule data does not meet a condition, the denoised small molecule data needs to be used as noisy small molecule data, and denoising processing is performed on the noisy small molecule data again until the condition is met, to obtain target small molecule data. After an experimental test is performed on the target small molecule data and the test passes, the target small molecule data may be used as drug data, thereby implementing drug research and development. Therefore, generation of the small molecule data is related to drug research and development. Based on this, how to determine the noise data in the noisy small molecule data becomes a problem urgently to be resolved.

An embodiment of this application provides a method for training a noise data determining model. The method may be applied to the foregoing implementation environment. Final noise data in to-be-processed noisy small molecule data may be determined, so that denoising processing can be performed on the to-be-processed noisy small molecule data based on the final noise data, thereby laying a foundation for generation of target small molecule data. A flowchart of a method for training a noise data determining model according to an embodiment of this application shown in FIG. 2 is used as an example. For ease of description, the terminal device 101 or the server 102 performing the method for training a noise data determining model in this embodiment of this application is referred to as an electronic device. The method may be performed by the electronic device. As shown in FIG. 2, the method includes operation 201 to operation 204.

Operation 201: Obtain sample noisy small molecule data and annotated noise data.

In this embodiment of this application, a sample noisy small molecule is a noisy small molecule, and the sample noisy small molecule includes a plurality of sample atoms, where any sample atom may be a noisy atom or an atom without noise. For any noisy atom, there is an error in information related to the atom and the error is out of an error range. For any atom without noise, there is no error in information related to the atom, or there is an error in the information related to the atom but the error is within an error range. Any sample atom has corresponding data, and information related to the sample atom may be described by using the data of the sample atom.

In one embodiment, the data of the sample atom may include data configured for describing a type of the sample atom. That is, the data of the sample atom includes type data of the sample atom. For example, if the sample atom is an oxygen atom, the type data of the sample atom is an element symbol O; if the sample atom is a carbon atom, the type data of the sample atom is an element symbol C; and if the sample atom is a nitrogen atom, the type data of the sample atom is an element symbol N.

The data of the sample atom may include data configured for describing a location of the sample atom, that is, the data of the sample atom includes location data of the sample atom. The location data of the sample atom may be three-dimensional coordinates of the sample atom, including an abscissa (usually represented by x), an ordinate (usually represented by y), and a vertical coordinate (usually represented by z). A location of the sample atom in a three-dimensional coordinate system is described by using the three coordinates.

The sample noisy small molecule includes the plurality of sample atoms, and data of the sample atoms forms the sample noisy small molecule data. In other words, the sample noisy small molecule data is small molecule data with noise data, and the sample noisy small molecule data includes the data of the plurality of sample atoms. The sample noisy small molecule data may include other data in addition to the data of the sample atoms. For example, the other data includes data configured for representing a small molecule type to which the sample noisy small molecule belongs.

In one embodiment, the sample noisy small molecule data may be represented as G={(aᵢ, rᵢ)}ᵢ₌₁ ^{N}, where N is a number of sample atoms, aᵢ is type data of a i^{th} sample atom, and rᵢ is location data of the i^{th} sample atom.

In one embodiment, an actual small molecule may be used as a sample small molecule, or a designed small molecule may be used as the sample small molecule. The sample small molecule is an effective small molecule that can bind to a sample protein. Data of atoms in the sample small molecule is obtained by analyzing information such as types and locations of the atoms in the sample small molecule, to obtain sample small molecule data. The sample small molecule data is data related to the small molecule without noise. A first time of noise processing may be performed on the sample small molecule data, to obtain small molecule data after the first time of noise processing; and a second time of noise processing is performed on the small molecule data after the first time of noise processing, to obtain small molecule data after the second time of noise processing. The rest is deduced by analogy. In other words, noise processing is performed T times on the sample small molecule data, so that small molecule data after the first time of noise processing to a T^{th} time of noise processing may be obtained, where T is a positive integer. The small molecule data after the T^{th} time of noise processing is initial noise data described below, and the sample small molecule data may be understood as small molecule data after a 0^{th} time of noise processing, namely, effective small molecule data described below.

FIG. 3 is a schematic diagram of noise processing and denoising processing according to an embodiment of this application. Noise processing is performed on small molecule data after a (t−1)^{th} time of noise processing once, to obtain small molecule data after a t^{th} time of noise processing. Through the principle, noise processing can be continuously performed on the small molecule data after the 0^{th} time of noise processing, and the small molecule data after the T^{th} time of noise processing is obtained through the T times of noise processing.

The small molecule data after the t^{th} time of noise processing may be used as the sample noisy small molecule data, and noise data when the t^{th} time of noise processing is performed on the small molecule data after the (t−1)^{th} time of noise processing, namely, noise data during the t^{th} time of noise processing, is used as the annotated noise data, where t is a positive integer greater than or equal to 1 and less than or equal to T. The annotated noise data may be understood as noise data obtained from the sample noisy small molecule data through annotation.

In one embodiment, when the T times of noise processing are performed on the small molecule data after the 0^{th} time of noise processing, T generally has a large value. n-m times of noise processing are performed on small molecule data after an m^{th} time of noise processing, so that small molecule data after an nth time of noise processing may be obtained, where both m and n are positive integers and less than or equal to T, and m is less than n. In this case, the small molecule data after the n^{th} time of noise processing may be used as the sample noisy small molecule data, and the annotated noise data is determined by using noise data during each of the n-m times of noise processing.

For example, when 1000 times of noise processing are performed on the small molecule data after the 0^{th} time of noise processing, small molecule data after a 1000^{th} time of noise processing is obtained. The small molecule data after the 1000^{th} time of noise processing, small molecule data after a 9990^{th} time of noise processing, . . . , small molecule data after a 10^{th} time of noise processing, and small molecule data after the 1^{st} time of noise processing each may be used as the sample noisy small molecule data in an interval sampling manner. When the small molecule data after the 1000^{th} time of noise processing is the sample noisy small molecule data, the annotated noise data is a sum of noise data during each of the 9990^{th} time of noise processing to the 1000^{th} time of noise processing. The rest is deduced by analogy. When the small molecule data after the 10^{th} time of noise processing is the sample noisy small molecule data, the annotated noise data is a sum of noise data during each of the 1^{st} time of noise processing to the 10^{th} time of noise processing.

Small molecule data after any time of noise processing is used as the sample noisy small molecule data, at least once of noise processing needs to be performed in a process of obtaining the sample noisy small molecule data, and a sum of noise data during each of the at least once of noise processing is used as the annotated noise data, so that predicted noise data determined by a neural network model is the sum of the noise data during each of the at least once of noise processing. The predicted noise data is removed from the sample noisy small molecule data, so that small molecule data after at least once of denoising processing is performed on the sample noisy small molecule data can be obtained, and efficiency of generating an effective small molecule is improved, thereby improving drug research and development efficiency.

Operation 202: Output a sample graph structure by using the neural network model based on the data of the plurality of sample atoms.

The sample graph structure includes a plurality of sample nodes and a plurality of sample edges, any sample node represents data of one sample atom, and any sample edge represents a distance between sample atoms corresponding to two sample nodes at two ends of the sample edge.

The sample noisy small molecule data may be inputted into the neural network model, and the neural network model constructs the sample graph structure based on the data of the sample atoms. The sample graph structure includes the plurality of sample nodes, and any sample node represents data of one sample atom. There may be an edge or no edge between sample nodes corresponding to any two sample atoms. When there is an edge between sample nodes corresponding to two sample atoms, the edge may be referred to as a sample edge, and the sample edge represents a distance between the two sample atoms.

A model structure, a model parameter, and the like of the neural network model are not limited in the embodiments of this application. For example, the neural network model is an initial network model. In this case, the model structure, the model parameter, and the like of the neural network model are exactly the same as a model structure, model parameter, and the like of the initial network model. In one embodiment, the initial network model includes at least one of a small molecule encoder, a protein encoder, a number-of-times encoder, a graph structure generator, a noise generator, or the like. Functions each are correspondingly described below, and details are not described herein again. Alternatively, the neural network model is a model obtained by performing at least once of training on the initial network model in the manner of operation 201 to operation 204. In this case, there is only a difference in model parameters between the neural network model and the initial network model, and the model structures of the two are the same.

An embodiment of this application provides Implementation A1. In Implementation A1, operation 202 includes operation 2021 to operation 2023.

Operation 2021: Perform feature extraction on the data of the plurality of sample atoms by using the neural network model, to obtain initial atomic features of the sample atoms.

In a possible implementation, the neural network model includes the small molecule encoder. The sample noisy small molecule data may be inputted into the small molecule encoder, and the small molecule encoder performs feature extraction on the data of the sample atoms, to obtain the initial atomic features of the sample atoms.

A model structure, a model parameter, and the like of the small molecule encoder are not limited in the embodiments of this application. For example, the small molecule encoder is an auto-encoder (AE) or a variational auto-encoder (VAE).

In one embodiment, data of any sample atom includes at least one of type data of the sample atom or location data of the sample atom. The small molecule encoder performs encoding processing on the type data of the sample atoms, to obtain a type feature of the sample atom. For example, the type data of the sample atom is an element symbol that can represent a type of the sample atom, and the small molecule encoder performs encoding processing such as one-hot encoding or multi-hot encoding on the element symbol, to obtain the type feature of the sample atom. The small molecule encoder determines a location feature of the sample atom based on the location data of the sample atom. For example, the location data of the sample atom is three-dimensional coordinates of the sample atom, and the small molecule encoder uses the three-dimensional coordinates of the sample atom as the location feature of the sample atom, or the small molecule encoder performs normalization processing on the three-dimensional coordinates of the sample atom, to obtain the location feature of the sample atom. The type feature of the sample atom or the location feature of the sample atom may be used as the initial atomic feature of the sample atom, or the type feature of the sample atom and the location feature of the sample atom are spliced to obtain the initial atomic feature of the sample atom.

Operation 2022: Obtain data of a sample protein, and perform feature extraction on the data of the sample protein by using the neural network model, to obtain a feature of the sample protein.

An actual protein may be used as the sample protein, or a designed protein may be used as the sample protein. The sample protein includes a plurality of atoms. Any atom has corresponding data, and information related to the atom is described by using the data of the atom. In one embodiment, data of any atom includes at least one of location data of the atom or type data of the atom. Data of the atom forms the data of the sample protein. The data of the sample protein may include other data in addition to the data of the atoms. For example, the other data includes data configured for representing a protein type to which the sample protein belongs.

In a possible implementation, the neural network model includes the protein encoder. The data of the sample protein may be inputted into the protein encoder, and the protein encoder performs feature extraction on the data of the atoms in the sample protein, to obtain features of the atoms in the sample protein.

A model structure, a model parameter, and the like of the protein encoder are not limited in the embodiments of this application. For example, the protein encoder is a VAE or a Schnet, where the Schnet is a variant of a deep tensor neural network (DTNN).

In one embodiment, the protein encoder performs encoding processing on type data of the atoms in the sample protein, to obtain type features of the atoms in the sample protein. The protein encoder determines location features of the atoms in the sample protein based on location data of the atoms in the sample protein. The type feature of any atom in the sample protein or the location feature of the atom may be used as the feature of the atom, or the type feature of the atom and the location feature of the atom are spliced to obtain the feature of the atom.

As used herein, the features of the atoms in the sample protein being obtained is equivalent to the feature of the sample protein being obtained. In other words, the features of the atoms in the sample protein may be used as the feature of the sample protein. Alternatively, convolution processing, normalization processing, canonical processing, or the like may be performed on the features of the atoms in the sample protein, to obtain the feature of the sample protein.

Operation 2023: Determine the sample graph structure by using the neural network model based on the initial atomic features of the sample atoms and the feature of the sample protein.

In a possible implementation, the neural network model may further include the graph structure generator. The initial atomic features of the sample atoms and the feature of the sample protein may be inputted into the graph structure generator, and the graph structure generator generates the sample graph structure.

In this embodiment of this application, the sample graph structure is generated based on the feature of the sample protein, so that when the predicted noise data is determined based on the sample graph structure, the predicted noise data is the noise data in the sample noisy small molecule data determined by the neural network model based on the sample protein. After denoising processing is performed on the sample noisy small molecule data based on the predicted noise data, a small molecule corresponding to obtained denoised small molecule data is more likely to bind to the sample protein. The higher a probability that the small molecule binds to the protein, the more likely the small molecule is to be a drug. Therefore, the sample graph structure is determined based on the feature of the sample protein, to determine the predicted noise data through the sample graph structure, so that data of the small molecule that can bind to the sample protein can be determined based on the predicted noise data, thereby obtaining the effective small molecule that can bind to the sample protein. This, in turn, can improve drug research and development efficiency. In this embodiment of this application, the data of the sample protein may be understood as a constraint condition for determining the noise data in the sample noisy small molecule data.

In one embodiment, the data of the sample protein is used as a constraint condition, to perform noise processing on the small molecule data after any time of noise processing. The noise process is shown in the following Formula (1).

$$q ( {G}^{1 : T} \vert {G}^{0} , {p}_{c t x} ) = {\prod }_{t = I}^{T} q ( {G}^{t} \vert {G}^{t - 1} , {p}_{c t x} ) \qquad Formula ( 1 )$$

In Formula (1), G⁰ represents the small molecule data after the 0^{th} time of noise processing, G^{t} represents the small molecule data after the t^{th} time of noise processing, G^{t-1} represents the small molecule data after the (t−1)^{th} time of noise processing, and G^{1:T} represents the small molecule data after the 1^{st} time of noise processing to the T^{th} time of noise processing; p_{ctx} represents the data of the sample protein; q(x) represents a function symbol of a noise processing function, where x is a variable; and Π represents a product symbol.

Also, in Formula (1), q(G^{1:T}|G⁰, p_{ctx}) represents the small molecule data after the 1^{st} time of noise processing to the T^{th} time of noise processing sequentially obtained by performing the T times of noise processing on the small molecule data after the 0^{th} time of noise processing by using the data of the sample protein as the condition; and q(G^{t}|G^{t-1}, p_{ctx}) represents the small molecule data after the t^{th} time of noise processing obtained by performing the t^{th} time of noise processing on the small molecule data after the (t−1)^{th} time of noise processing by using the data of the sample protein as the condition.

In one embodiment, the small molecule data after the t^{th} time of noise processing meets the following Formula (2).

$$q ( {G}^{t} \vert {G}^{t - 1} , {p}_{c t x} ) = 𝒩 ( {G}^{t} ; \sqrt{1 - {β}_{t}} {G}^{t - 1} , {β}_{t} I ) \qquad Formula ( 2 )$$

In Formula (2), [symbol] represents a function symbol of a normal distribution function. Generally, the normal distribution function is [symbol] (0, 1), where I is a parameter of the normal distribution function. β₁ . . . β_{T} are fixed variance parameters, and βₜ is a t^{th} variance parameter. In one embodiment, the t^{th} variance parameter meets: at ϑₜ=1−βₜ, ϑ̅ₜ=Πₛ₌₁^{t}ϑₛ. In this embodiment of this application, Formula (2) represents that the small molecule data after the t^{th} time of noise processing meets the normal distribution function [symbol] (G^{t}; \sqrt{1−βₜ}G^{t-1}, βₜI).

Because the data of the sample protein is used as the constraint condition when noise processing is performed on the small molecule data after any time of noise processing, the data of the sample protein also needs to be used as a constraint condition, to perform denoising processing on the small molecule data after any time of noise processing. The denoising process is shown in the following Formula (3).

$${p}_{θ} ( {G}^{0 : T - 1} \vert {G}^{T} , {p}_{c t x} ) = {\prod }_{t = 1}^{T} {p}_{θ} ( {G}^{t - 1} \vert {G}^{t} , {p}_{c t x} ) \qquad Formula ( 3 )$$

In Formula (3), G⁰ represents the small molecule data after the 0^{th} time of noise processing, G^{t} represents the small molecule data after the t^{th} time of noise processing, G^{t-1} represents the small molecule data after the (t−1)^{th} time of noise processing, and G^{0:T-1} represents small molecule data after the 0^{th} time of noise processing to a (T−1)^{th} time of noise processing; p_{ctx} represents the data of the sample protein; P_{θ}( [symbol] ⁰) represents a function symbol of a denoising processing function, where [symbol] ⁰ is a variable; and Π represents a product symbol.

Also, in Formula (3), P_{θ}(G^{0:T-1}|G^{T}, p_{ctx}) represents the small molecule data after the 0^{th} time of noise processing to the (T−1)^{th} time of noise processing sequentially obtained by performing T times of denoising processing on the small molecule data after the T^{th} time of noise processing by using the data of the sample protein as the condition; and p_{θ}(G^{t-1}|G^{t}, p_{ctx}) represents the small molecule data after the (t−1)^{th} time of noise processing obtained by performing a t^{th} time of denoising processing on the small molecule data after the t^{th} time of noise processing by using the data of the sample protein as the condition.

In one embodiment, the small molecule data after the (t−1)^{th} time of noise processing meets the following Formula (4).

$${p}_{θ} ( {G}^{t - 1} \vert {G}^{t} , {p}_{c t x} ) = 𝒩 ( {G}^{t - 1} ; {μ}_{θ} ( {G}^{t} , {p}_{c t x} , t ) , {σ}_{t}^{2} I ) \qquad Formula ( 4 )$$

In Formula (4), [symbol] represents a function symbol of a normal distribution function. Generally, the normal distribution function is [symbol] (0, I), where I is a parameter of the normal distribution function. Also, in Formula (4), μ_{θ} is a mean value of the small molecule data after the t^{th} time of noise processing that meets a distribution; and σₜ is a variance value, and may be arbitrarily set data. In this embodiment of this application, Formula (4) represents that the small molecule data after the (t−1)^{th} time of noise processing meets the normal distribution function [symbol] (G^{t-1}; μ_{θ}(G^{t}, p_{ctx}, t), σₜ²I), where μ_{θ} is a parameter that needs to be learned by the neural network model in this embodiment of this application. In a process of training the neural network model, maximum likelihood estimation needs to be performed on p_{θ}( [symbol] ⁰).

In one embodiment, operation 2023 includes: fusing, for any sample atom, the initial atomic feature of the any sample atom and the feature of the sample protein by using the neural network model, to obtain a first atomic feature of the any sample atom; determining a first distance between each two sample atoms based on first atomic features of the sample atoms; and determining the sample graph structure based on the first atomic features of the sample atoms and the first distance between each two sample atoms.

The initial atomic features of the sample atoms and the feature of the sample protein are fused to obtain the first atomic features of the sample atoms, so that the first atomic features of the sample atoms can be expressed based on the feature of the sample protein, and then the sample graph structure is determined based on the feature of the sample protein, to determine the noise data in the sample noisy small molecule data based on the sample protein. In this way, the data of the small molecule that can bind to the sample protein can be determined based on the predicted noise data, thereby obtaining the effective small molecule that can bind to the sample protein. This, in turn, may improve drug research and development efficiency.

The neural network model performs any fusion processing such as splicing, addition, or multiplication on the initial atomic feature of any sample atom and the feature of the sample protein, to obtain the first atomic feature of the sample atom; and determines the first distance between any two sample atoms based on first atomic features of the two sample atoms according to a distance formula. The distance formula is not limited in the embodiments of this application. For example, the distance formula is a cosine distance formula, a cross-entropy distance formula, a relative entropy distance formula, or the like. Through the distance formula, the first distance between each two sample atoms can be determined.

In one embodiment, the first atomic feature of any sample atom is used as a sample node, and the first distance between any two sample atoms is used as an edge between sample nodes corresponding to the two sample atoms. In this way, the sample nodes and the edge between each two sample nodes can be determined, to obtain the sample graph structure.

Alternatively, the first atomic feature of any sample atom is used as a sample node. For any two sample nodes, if the first distance between sample atoms corresponding to the any two sample nodes is greater than a distance threshold, it is determined that there is no edge between the two sample nodes; and if the first distance between the sample atoms corresponding to the any two sample nodes is not greater than the distance threshold, the first distance between the sample atoms corresponding to the two sample nodes is determined as an edge between the two sample nodes. In this way, the sample nodes and the edge between any two sample nodes can be determined, to obtain the sample graph structure. The distance threshold is not limited in the embodiments of this application. In one embodiment, the distance threshold is a value set according to manual experience, or the distance threshold is a maximum distance at which an interaction force can exist between two sample atoms.

In Implementation A1, the sample graph structure is determined according to the initial atomic features of the sample atoms and the feature of the sample protein. An embodiment of this application further provides Implementation A2. In Implementation A2, the sample graph structure may be determined only according to initial atomic features of the sample atoms.

For example, an initial distance between each two sample atoms is determined based on the initial atomic features of the sample atoms; and the sample graph structure is determined based on the initial atomic features of the sample atoms and the initial distance between each two sample atoms. A manner of determining the initial distance between two sample atoms is similar to a manner of determining the first distance between two sample atoms, and a manner of determining the sample graph structure based on the initial atomic features of the sample atoms and the initial distance between each two sample atoms is similar to a manner of “determining the sample graph structure based on the first atomic features of the sample atoms and the first distance between each two sample atoms”. Details are not described herein again.

An embodiment of this application further provides a manner of determining the sample graph structure different from Implementation A1 and Implementation A2, as shown in the following Implementation A3.

In Implementation A3, the sample noisy small molecule data is initial noise data or is obtained by performing at least once of denoising processing on the initial noise data.

It has been described above that, the initial noise data may be considered as the small molecule data after the T^{th} time of noise processing. A first time of denoising processing is performed on the small molecule data after the T^{th} time of noise processing, to obtain small molecule data after a (T−1)^{th} time of noise processing; and a second time of denoising processing is performed on the small molecule data after the (T−1)^{th} time of noise processing, to obtain small molecule data after a (T-2)^{th} time of noise processing. The rest is deduced by analogy. In other words, T times of denoising processing are performed on the small molecule data after the T^{th} time of noise processing, to obtain the small molecule data after the 0^{th} time of noise processing, namely, the sample small molecule data described above.

Referring to FIG. 3, denoising processing is performed on the small molecule data after the t^{th} time of noise processing once, to obtain the small molecule data after the (t−1)^{th} time of noise processing. In this way, denoising processing can be continuously performed on the small molecule data after the T^{th} time of noise processing, and the small molecule data after the 0^{th} time of noise processing is obtained through the T times of denoising processing.

The small molecule data after the t^{th} time of noise processing may be used as the sample noisy small molecule data, where t is a positive integer greater than or equal to 1 and less than or equal to T.

In Implementation A3, operation 202 includes operation 2024 and operation 2025.

Operation 2024: Obtain sample number-of-times-of-denoising information, where the sample number-of-times-of-denoising information represents a number of times of denoising processing performed to change the initial noise data to the sample noisy small molecule data.

In this embodiment of this application, the T times of noise processing are performed on the small molecule data after the 0^{th} time of noise processing, to sequentially obtain the small molecule data after the first time of noise processing to the T^{th} time of noise processing. The T times of denoising processing are performed on the small molecule data after the T^{th} time of noise processing, to sequentially obtain small molecule data after the (T−1)^{th} time of noise processing to the 0^{th} time of noise processing. Therefore, the t^{th} time of noise processing and a (T−t)^{th} time of denoising processing are two inverse processes.

The sample noisy small molecule data being the small molecule data after the t^{th} time of noise processing indicates that t times of noise processing are required to change the small molecule data after the 0^{th} time of noise processing to the small molecule data after the t^{th} time of noise processing. Based on this, T−t times of denoising processing are required to change the small molecule data after the T^{th} time of noise processing to the small molecule data after the t^{th} time of noise processing, and the initial noise data may be considered as the small molecule data after the T^{th} time of noise processing. Therefore, the sample number-of-times-of-denoising information is T−t.

Operation 2025: Determine the sample graph structure by using the neural network model based on the sample number-of-times-of-denoising information and the data of the plurality of sample atoms.

In this embodiment of this application, the neural network model may determine the sample graph structure based on the sample number-of-times-of-denoising information and the data of the plurality of sample atoms according to at least Implementation B1 or Implementation B2 described below.

The sample number-of-times-of-denoising information may represent the number of times of denoising processing performed to change the initial noise data to the sample noisy small molecule data. The sample graph structure is determined based on the sample number-of-times-of-denoising information and the data of the plurality of sample atoms, so that the predicted noise data can be more quickly obtained based on the sample graph structure, thereby improving the drug research and development efficiency.

In Implementation B1, operation 2025 includes operation C1 and operation C3.

Operation C1: Perform feature extraction on the sample number-of-times-of-denoising information by using the neural network model, to obtain a sample number-of-times-of-denoising feature.

In a possible implementation, the neural network model may further include the number-of-times encoder. The sample number-of-times-of-denoising information is inputted into the number-of-times encoder, and the number-of-times encoder performs feature extraction on the sample number-of-times-of-denoising information, to obtain the sample number-of-times-of-denoising feature. A model structure, a model parameter, and the like of the number-of-times encoder are not limited in the embodiments of this application. For example, the number-of-times encoder is a multilayer perceptron, an AE, or the like. In one embodiment, the number-of-times encoder performs encoding processing such as one-hot encoding or multi-hot encoding on the sample number-of-times-of-denoising information, to obtain the sample number-of-times-of-denoising feature.

Operation C2: Perform feature extraction on the data of the plurality of sample atoms respectively by using the neural network model, to obtain initial atomic features of the sample atoms.

The small molecule encoder in the neural network model may perform feature extraction on the data of the sample atoms, to obtain the initial atomic features of the sample atoms. Actions used to perform operation 2021 may be similarly used to perform operation C2. Details are not described herein again.

Operation C3: Determine the sample graph structure by using the neural network model based on the sample number-of-times-of-denoising feature and the initial atomic features of the sample atoms.

The graph structure generator in the neural network model may determine the sample graph structure based on the sample number-of-times-of-denoising feature and the initial atomic features of the sample atoms according to at least Implementation D1 or Implementation D2.

In Implementation D1, operation C3 includes: fusing, for any sample atom, the initial atomic feature of the any sample atom and the sample number-of-times-of-denoising feature by using the neural network model, to obtain a second atomic feature of the any sample atom; determining a second distance between each two sample atoms based on second atomic features of the sample atoms; and determining the sample graph structure based on the second atomic features of the sample atoms and the second distance between each two sample atoms.

The neural network model performs any fusion processing such as splicing, addition, or multiplication on the initial atomic feature of any sample atom and the sample number-of-times-of-denoising feature, to obtain the second atomic feature of the sample atom; and determines the second distance between any two sample atoms based on second atomic features of the two sample atoms according to a distance formula. Through the manner, the second distance between each two sample atoms can be determined.

In one embodiment, the second atomic feature of any sample atom is used as a sample node, and the second distance between any two sample atoms is used as an edge between sample nodes corresponding to the two sample atoms. Through the manner, the sample nodes and the edge between each two sample nodes can be determined, to obtain the sample graph structure.

Alternatively, the second atomic feature of any sample atom is used as a sample node. For any two sample nodes, if the second distance between sample atoms corresponding to the any two sample nodes is greater than a distance threshold, it is determined that there is no edge between the two sample nodes; and if the second distance between the sample atoms corresponding to the any two sample nodes is not greater than the distance threshold, the second distance between the sample atoms corresponding to the two sample nodes is determined as an edge between the two sample nodes. Through the manner, the sample nodes and the edge between any two sample nodes can be determined, to obtain the sample graph structure.

In Implementation D2, operation C3 includes: fusing, for any sample atom, the initial atomic feature of the any sample atom, the sample number-of-times-of-denoising feature, and a feature of a sample protein by using the neural network model, to obtain a third atomic feature of the any sample atom; determining a third distance between each two sample atoms based on third atomic features of the sample atoms; and determining the sample graph structure based on the third atomic features of the sample atoms and the third distance between each two sample atoms.

The neural network model performs any fusion processing such as splicing, addition, or multiplication on the initial atomic feature of any sample atom, the sample number-of-times-of-denoising feature, and the feature of the sample protein, to obtain the third atomic feature of the sample atom; and determines the third distance between any two sample atoms based on third atomic features of the two sample atoms according to a distance formula. In this way, the third distance between each two sample atoms can be determined.

In one embodiment, the third atomic feature of any sample atom is used as a sample node, and the third distance between any two sample atoms is used as an edge between sample nodes corresponding to the two sample atoms. In this way, the sample nodes and the edge between each two sample nodes can be determined, to obtain the sample graph structure.

Alternatively, the third atomic feature of any sample atom is used as a sample node. For any two sample nodes, if the third distance between sample atoms corresponding to the any two sample nodes is greater than a distance threshold, it is determined that there is no edge between the two sample nodes; and if the third distance between the sample atoms corresponding to the any two sample nodes is not greater than the distance threshold, the third distance between the sample atoms corresponding to the two sample nodes is determined as an edge between the two sample nodes. In this way, the sample nodes and the edge between any two sample nodes can be determined, to obtain the sample graph structure.

In Implementation B2, Operation 2025 includes: determining sample number-of-times-of-noise-processing information based on the sample number-of-times-of-denoising information, and performing feature extraction on the sample number-of-times-of-noise-processing information by using the neural network model, to obtain a sample number-of-times-of-noise-processing feature; performing feature extraction on the data of the plurality of sample atoms by using the neural network model, to obtain initial atomic features of the sample atoms; and determining the sample graph structure by using the neural network model based on the sample number-of-times-of-noise-processing feature and the initial atomic features of the sample atoms.

The t^{th} time of noise processing and the (T−t)^{th} time of denoising processing are two inverse processes. Therefore, when it is determined that the sample number-of-times-of-denoising information is T−t, the sample number-of-times-of-noise-processing information may be determined as t based on the sample number-of-times-of-denoising information.

The sample number-of-times-of-noise-processing information is inputted into the number-of-times encoder in the neural network model, and the number-of-times encoder performs feature extraction on the sample number-of-times-of-noise-processing information, to obtain the sample number-of-times-of-noise-processing feature. In one embodiment, the number-of-times encoder performs encoding processing such as one-hot encoding or multi-hot encoding on the sample number-of-times-of-noise-processing information, to obtain the sample number-of-times-of-noise-processing feature.

In addition, the small molecule encoder in the neural network model may perform feature extraction on the data of the sample atoms, to obtain the initial atomic features of the sample atoms. Actions used to perform operation 2021 may be similarly used here to determine the initial atomic features. Details are not described herein again.

Next, the neural network model determines the sample graph structure based on the sample number-of-times-of-noise-processing feature and the initial atomic features of the sample atoms.

In one embodiment, for any sample atom, the neural network model fuses the initial atomic feature of the any sample atom and the sample number-of-times-of-noise-processing feature, to obtain a fourth atomic feature of the any sample atom; determines a fourth distance between each two sample atoms based on fourth atomic features of the sample atoms; and determines the sample graph structure based on the fourth atomic features of the sample atoms and the fourth distance between each two sample atoms. Actions used to perform Implementation D1 may be similarly used here to determine the sample graph structure and/or the fourth distance. Implementation principles of the two are similar, and details are not described herein again.

Alternatively, for any sample atom, the neural network model fuses the initial atomic feature of the any sample atom, the sample number-of-times-of-noise-processing feature, and a feature of a sample protein, to obtain a fifth atomic feature of the any sample atom; determines a fifth distance between each two sample atoms based on fifth atomic features of the sample atoms; and determines the sample graph structure based on the fifth atomic features of the sample atoms and the fifth distance between each two sample atoms. Actions used to perform Implementation D2 may be similarly used here to determine the fifth distance and/or the sample graph structure. Implementation principles of the two are similar, and details are not described herein again.

In Implementation A3, the sample number-of-times-of-denoising information is directly obtained, and the sample graph structure is constructed by using the sample number-of-times-of-denoising information; or the sample number-of-times-of-noise-processing information is determined based on the sample number-of-times-of-denoising information, and the sample graph structure is constructed based on the sample number-of-times-of-noise-processing information. During application, based on the principle of Implementation A3, the sample number-of-times-of-noise-processing information may be directly obtained, and the sample graph structure is constructed by using the sample number-of-times-of-noise-processing information; or the sample number-of-times-of-denoising information is determined based on the sample number-of-times-of-noise-processing information, and the sample graph structure is constructed based on the sample number-of-times-of-denoising information. Details are not described herein again.

Operation 203: Perform prediction on the sample graph structure by using the neural network model, to obtain predicted noise data.

In a possible implementation, the neural network model may further include the noise generator. The sample graph structure is inputted into the noise generator, and the noise generator determines the predicted noise data based on the sample graph structure, where the predicted noise data is noise data obtained through prediction.

In one embodiment, the noise generator includes a graph encoder and an activation layer, where functions of the graph encoder and the activation layer are correspondingly described below. Details are not described herein again. A network structure, a network parameter, and the like of the graph encoder are not limited in the embodiments of this application. For example, the graph encoder may be a graph auto-encoder (GAE), a graph variational auto-encoder (GVAE), or the like. A network structure, a network parameter, and the like of the activation layer are also not limited in the embodiments of this application. For example, the activation layer may be a rectified linear unit (ReLU), a Sigmoid growth curve (namely, a Sigmoid function), or the like.

In Implementation E1, operation 203 includes operation 2031 and operation 2033.

Operation 2031: Perform feature extraction on the sample graph structure by using the neural network model, to obtain to-be-processed atomic features of the sample atoms.

In this embodiment of this application, the sample graph structure may be inputted into the graph encoder, and the graph encoder performs at least once of updating processing on the sample graph structure, to obtain an updated sample graph structure. Sample nodes in the updated sample graph structure are the to-be-processed atomic features of the sample atoms.

It has been described above that, the sample graph structure includes the plurality of sample nodes and the plurality of sample edges; any sample node is an initial atomic feature of one sample atom, or any sample node is any one of the first atomic feature to the fifth atomic feature of one sample atom; and any sample node is connected to another sample node through one sample edge.

When updating is performed on the sample graph structure once, for any sample node, the sample node may be updated by using the sample node and another sample node connected to the sample node through a sample edge; or the sample node may be updated by using the sample node, sample edges having one end being the sample node, and another sample node connected to the sample node through a sample edge. Through the manner, the sample nodes in the sample graph structure may be updated, to obtain the updated sample graph structure.

The sample nodes in the updated sample graph structure are used as the to-be-processed atomic features of the sample atoms. Alternatively, the updated sample graph structure is used as a sample graph structure, the sample nodes in the sample graph structure are updated again to obtain an updated sample graph structure, and the sample nodes in the updated sample graph structure are used as the to-be-processed atomic features of the sample atoms.

Operation 2032: Determine, based on the to-be-processed atomic features of the sample atoms, at least one of predicted type noise data or predicted location noise data by using the neural network model, the predicted type noise data being noise data related to types of the sample atoms obtained through prediction, and the predicted location noise data being noise data related to locations of the sample atoms obtained through prediction.

In this embodiment of this application, the to-be-processed atomic features of the sample atoms may be inputted into the activation layer in the noise generator, and the activation layer performs activation processing on the to-be-processed atomic features of the sample atoms, to obtain the predicted type noise data and/or the predicted location noise data.

In one embodiment, the activation layer performs activation processing on the to-be-processed atomic features of the sample atoms, to obtain type noise data of the sample atoms, where type noise data of any sample atom is noise data related to the type of the sample atom obtained through prediction. The predicted type noise data includes the type noise data of the sample atoms.

Similarly, the activation layer performs activation processing on the to-be-processed atomic features of the sample atoms, to obtain location noise data of the sample atoms, where location noise data of any sample atom is noise data related to the location of the sample atom obtained through prediction. The predicted location noise data includes the location noise data of the sample atoms.

Operation 2033: Use the at least one of the predicted type noise data or the predicted location noise data as the predicted noise data.

In this embodiment of this application, the predicted type noise data or the predicted location noise data may be used as the predicted noise data. Alternatively, the predicted type noise data and the predicted location noise data may be used as the predicted noise data, or the predicted type noise data or the predicted location noise data may be used as the predicted noise data.

In Implementation E2, operation 203 includes operation 2034 to operation 2036.

Operation 2034: Determine, for any sample edge included in the sample graph structure, the any sample edge as a first edge if a distance represented by the any sample edge is not greater than a reference distance; and delete, by using the neural network model, the first edge from the plurality of sample edges included in the sample graph structure, to obtain a first graph structure.

The reference distance is not limited in the embodiments of this application. For example, the reference distance is a value set according to manual experience, or the reference distance is not less than a distance exerted by a chemical bond and not greater than a distance exerted by a van der Waals force. For example, the distance exerted by the chemical bond is less than the distance exerted by the van der Waals force. Generally, the distance exerted by the chemical bond is less than 2 angstroms (unit: Å), and the distance exerted by the van der Waals force is greater than 2 angstroms. In this case, the reference distance may be determined as 2 angstroms.

It has been described above that, any sample edge is the initial distance between sample atoms corresponding to two sample nodes at two ends of the sample edge, or any sample edge is any one of the first distance to the fifth distance between sample atoms corresponding to two sample nodes at two ends of the sample edge. In other words, any sample edge represents a distance between sample atoms corresponding to two sample nodes at two ends of the sample edge.

If the distance represented by any sample edge included in the sample graph structure is not greater than the reference distance, the sample edge is determined as the first edge, and the graph structure generator in the neural network model deletes the first edge from the sample graph structure. Through the manner, at least one first edge can be deleted from the sample graph structure, to obtain the first graph structure.

The foregoing first graph structure is obtained by deleting the at least one first edge from the sample graph structure. During application, there may be another manner of generating the first graph structure. For example, when the sample graph structure is constructed, if a distance between sample atoms corresponding to any two sample nodes (the distance may be any one of the first distance to the fifth distance) is greater than the reference distance, the distance between the sample atoms corresponding to the two sample nodes is determined as an edge between the two sample nodes; and if the distance between the sample atoms corresponding to the any two sample nodes is not greater than the reference distance, it is determined that there is no edge between the two sample nodes. Through the manner, the constructed sample graph structure is the first graph structure.

When the reference distance is not less than the distance exerted by the chemical bond and not greater than the distance exerted by the van der Waals force, a sample edge related to the chemical bond in the sample graph structure may be deleted by deleting the at least one first edge from the sample graph structure, so that the first graph structure includes a sample edge related to the van der Waals force.

Operation 2035: Determine first noise data by using the neural network model based on the first graph structure.

The first graph structure includes the sample edge related to the van der Waals force. Therefore, the neural network model determines the first noise data based on the first graph structure, so that the first noise data is determined based on the sample edge related to the van der Waals force. Because the first noise data is obtained by analyzing the van der Waals force in the sample noisy small molecule, the first noise data is related to a single factor, namely, the van der Waals force. In this way, the model can focus on learning a mapping relationship between the noise data and the van der Waals force, thereby improving accuracy of determining the noise data by the model. In other words, accuracy of the first noise data is high.

In this embodiment of this application, the first graph structure may be inputted into the noise generator, and the noise generator determines the first noise data based on the first graph structure, where the first noise data is noise data obtained through prediction.

In one embodiment, operation 2035 includes: performing feature extraction on the first graph structure by using the neural network model, to obtain sixth atomic features of the sample atoms; determining, based on the sixth atomic features of the sample atoms, at least one of first type noise data or first location noise data by using the neural network model, the first type noise data being noise data related to the types of the sample atoms obtained through prediction, and the first location noise data being noise data related to the locations of the sample atoms obtained through prediction; and using the at least one of the first type noise data or the first location noise data as the first noise data.

In this embodiment of this application, the first graph structure may be inputted into the graph encoder, and the graph encoder performs at least once of updating processing on the first graph structure, to obtain an updated first graph structure. Sample nodes in the updated first graph structure are the sixth atomic features of the sample atoms. A manner of updating the first graph structure is similar to a manner of updating the sample graph structure as described for operation 2031. Details are not described herein again.

In this embodiment of this application, the sixth atomic features of the sample atoms may be inputted into the activation layer in the noise generator, and the activation layer performs activation processing on the sixth atomic features of the sample atoms, to obtain the first type noise data and/or the first location noise data. A manner of determining the first type noise data is similar to a manner of determining the predicted type noise data, and a manner of determining the first location noise data is similar to a manner of determining the predicted location noise data. Details are not described herein again.

The first type noise data includes type noise data of the sample atoms, where type noise data of any sample atom is noise data related to the type of the sample atom obtained through prediction. Similarly, the first location noise data includes location noise data of the sample atoms, where location noise data of any sample atom is noise data related to the location of the sample atom obtained through prediction.

In this embodiment of this application, the first type noise data or the first location noise data may be used as the first noise data. Alternatively, the first type noise data and the first location noise data may be used as the first noise data, or the first type noise data or the first location noise data may be used as the first noise data.

Operation 2036: Determine the predicted noise data based on the first noise data.

In this embodiment of this application, the first noise data may be used as the predicted noise data, or the first noise data is multiplied by a corresponding weight to obtain the predicted noise data.

In Implementation E3, operation 203 includes operation 2037 to operation 2039.

Operation 2037: Determine, for any sample edge included in the sample graph structure, the any sample edge as a second edge if a distance represented by the any sample edge is greater than a reference distance; and delete, by using the neural network model, the second edge from the plurality of sample edges included in the sample graph structure, to obtain a second graph structure.

If the distance represented by any sample edge included in the sample graph structure is greater than the reference distance, the sample edge is determined as the second edge, and the graph structure generator in the neural network model deletes the second edge from the sample graph structure. In this way, at least one second edge can be deleted from the sample graph structure, to obtain the second graph structure.

The foregoing second graph structure is obtained by deleting the at least one second edge from the sample graph structure. During application, there may be another way to generate the second graph structure. For example, when the sample graph structure is constructed, if a distance between sample atoms corresponding to any two sample nodes (the distance may be any one of the first distance to the fifth distance) is greater than the reference distance, it is determined that there is no edge between the two sample nodes; and if the distance between the sample atoms corresponding to the any two sample nodes is not greater than the reference distance, the distance between the sample atoms corresponding to the two sample nodes is determined as an edge between the two sample nodes. In this way, the constructed sample graph structure is the second graph structure.

When the reference distance is not less than the distance exerted by the chemical bond and not greater than the distance exerted by the van der Waals force, a sample edge related to the van der Waals force in the sample graph structure may be deleted by deleting the at least one second edge from the sample graph structure, so that the second graph structure includes a sample edge related to the chemical bond.

Operation 2038: Determine second noise data by using the neural network model based on the second graph structure.

The second graph structure includes the sample edge related to the chemical bond. Therefore, the neural network model determines the second noise data based on the second graph structure, so that the second noise data is determined based on the sample edge related to the chemical bond. Because the second noise data is obtained by analyzing the chemical bond in the sample noisy small molecule, the second noise data is related to a single factor, namely, the chemical bond. In this way, the model can focus on learning a mapping relationship between the noise data and the chemical bond, thereby improving accuracy of determining the noise data by the model. In other words, accuracy of the second noise data is high.

In this embodiment of this application, the second graph structure may be inputted into the noise generator, and the noise generator determines the second noise data based on the second graph structure, where the second noise data is predicted noise data.

In one embodiment, operation 2038 includes: performing feature extraction on the second graph structure by using the neural network model, to obtain seventh atomic features of the sample atoms; determining, based on the seventh atomic features of the sample atoms, at least one of second type noise data or second location noise data by using the neural network model, the second type noise data being noise data related to the types of the sample atoms obtained through prediction, and the second location noise data being noise data related to the locations of the sample atoms obtained through prediction; and using the at least one of the second type noise data or the second location noise data as the second noise data.

In this embodiment of this application, the second graph structure may be inputted into the graph encoder, and the graph encoder performs at least once of updating processing on the second graph structure, to obtain an updated second graph structure. Sample nodes in the updated second graph structure are the seventh atomic features of the sample atoms. Updating the second graph structure may be performed in a similar way as updating the sample graph structure, as described for operation 2031. Details are not described herein again.

In this embodiment of this application, the seventh atomic features of the sample atoms may be inputted into the activation layer in the noise generator, and the activation layer performs activation processing on the seventh atomic features of the sample atoms, to obtain the second type noise data and/or the second location noise data. Determining the second type noise data may be performed in a similar way as determining the predicted type noise data, and determining the second location noise data may be performed in a similar way as determining the predicted location noise data. Details are not described herein again.

The second type noise data includes type noise data of the sample atoms, where type noise data of any sample atom is noise data related to the type of the sample atom obtained through prediction. Similarly, the second location noise data includes location noise data of the sample atoms, where location noise data of any sample atom is noise data related to the location of the sample atom obtained through prediction.

The second type noise data or the second location noise data may be used as the second noise data. Alternatively, the second type noise data and the second location noise data may be used as the second noise data, or the second type noise data or the second location noise data may be used as the second noise data.

Operation 2039: Determine the predicted noise data based on the second noise data.

In this embodiment of this application, the second noise data may be used as the predicted noise data, or the second noise data is multiplied by a corresponding weight to obtain the predicted noise data.

In Implementation E4, operation 203 includes: determining, for any sample edge included in the sample graph structure, the any sample edge as a first edge if a distance represented by the any sample edge is not greater than a reference distance; deleting, by using the neural network model, the first edge from the plurality of sample edges included in the sample graph structure, to obtain a first graph structure; determining first noise data by using the neural network model based on the first graph structure; determining, for any sample edge included in the sample graph structure, the any sample edge as a second edge if a distance represented by the any sample edge is greater than the reference distance; deleting, by using the neural network model, the second edge from the plurality of sample edges included in the sample graph structure, to obtain a second graph structure; determining second noise data by using the neural network model based on the second graph structure; and determining the predicted noise data based on the first noise data and the second noise data.

In this embodiment of this application, the first noise data may be determined according to content of Implementation E2, and the second noise data may be determined according to content of Implementation E3. The first noise data and the second noise data may be determined as the predicted noise data, or operation processing such as weighted averaging or weighted summation is performed on the first noise data and the second noise data, to obtain the predicted noise data.

In one embodiment, the first noise data includes first type noise data and first location noise data, and the second noise data includes second type noise data and second location noise data. Operation processing such as weighted summation or weighted averaging is performed on the first type noise data and the second type noise data, to obtain predicted type noise data. Operation processing such as weighted summation or weighted averaging is performed on the first location noise data and the second location noise data, to obtain predicted location noise data. The predicted noise data includes the predicted type noise data and the predicted location noise data.

Operation 204: Train the neural network model based on the predicted noise data and the annotated noise data, to obtain a noise data determining model, the noise data determining model being configured to determine final noise data based on to-be-processed noisy small molecule data.

In this embodiment of this application, a loss of the neural network model may be determined based on the predicted noise data and the annotated noise data. Training is performed on the neural network model based on the loss of the neural network model, to obtain a trained neural network model.

If the trained neural network model meets a training end condition, the trained neural network model is used as the noise data determining model. If the trained neural network model does not meet the training end condition, the trained neural network model is used as a neural network model, and the neural network model is trained according to operation 201 to operation 204 until the training end condition is met, to obtain the noise data determining model.

The training end condition is not limited in the embodiments of this application. For example, the training end condition is met when a number of times of training is reached, such as 500 times or 1000 times, as examples. Alternatively, the training end condition is met when a difference between a loss of a neural network model obtained through current training and a loss of a neural network model obtained through previous training falls within a set range. Alternatively, the training end condition is met when a gradient of a loss of a neural network model obtained through current training falls within a set range.

In one embodiment, the loss of the neural network model may be determined based on the predicted noise data and the annotated noise data according to the following Formula (5).

$${ℒ}_{E L B O} = {\sum }_{t = 1}^{T} {γ}_{T} {E}_{\{ {G}^{0} \} \sim q ( {G}^{0} ) , ϵ \sim 𝒩 ( 0 , I )} [ {‖ ϵ - {ϵ}_{θ} ( {G}_{t} , {p}_{ctx} , t ) ‖}_{2}^{2} ] \qquad Formula ( 5 )$$

In Formula (5), [symbol] _{ELBO} represents the loss of the neural network model; T represents a total number of times of denoising processing; Υₜ is a hyperparameter; ϵ represents the annotated noise data, where the annotated noise data may be designed to obey a normal distribution; ϵ_{θ}(Gₜ, p_{ctx}, t) represents the predicted noise data, where Gₜ represents the small molecule data after the t^{th} time of noise processing; and p_{ctx} represents the data of the sample protein. Also, in Formula (5), ∥ϵ−ϵ_{θ}(Gₜ, p_{ctx}, t)∥₂² represents calculating a mean square error between the annotated noise data and the predicted noise data; E is an averaging symbol; {G⁰}˜q(G⁰) represents that the small molecule data after the 0^{th} time of noise processing during noise processing is the same as the small molecule data after the 0^{th} time of noise processing during denoising processing; and ϵ˜ [symbol] (0, I) represents that the annotated noise data confirms to the normal distribution function [symbol] (0, I), where I is the parameter of the normal distribution function.

A derivation process of Formula (5) is shown in Formula (6).

$$E [ log {p}_{θ} ( {G}^{0} \vert {p}_{c t x} ) ] = E [ log {E}_{q ( {G}^{1 : T} \vert {G}^{0} , {p}_{c t x} )} \frac{{p}_{θ} ( {G}^{0 : T} \vert {p}_{c t x} )}{q ( {G}^{1 : T} \vert {G}^{0} , {p}_{c t x} )} ] \geq - {E}_{q} [ {\sum }_{t = 1}^{T} {D}_{K L} ( q ( {G}^{t - 1} \vert {G}^{t} , {G}^{0} , {p}_{c t x} ) ‖ {p}_{θ} ( {G}^{t - 1} \vert {G}^{t} , {p}_{c t x} ) ) ] := - {ℒ}_{B L B O} \qquad Formula ( 6 )$$

In Formula (6), E is an averaging symbol; log is a logarithmic symbol; p_{θ}(G⁰|p_{ctx}) represents the small molecule data after the 0^{th} time of noise processing obtained through denoising processing by using the data of the sample protein as the constraint condition; q(G^{1:T}|G⁰, p_{ctx}) represents the small molecule data after the first time of noise processing to the T^{th} time of noise processing sequentially obtained by performing the T times of noise processing on the small molecule data after the 0th time of noise processing by using the data of the sample protein as the constraint condition; p_{θ}(G^{0:T}|p_{ctx}) represents small molecule data after the 0^{th} time of noise processing to the T^{th} time of noise processing sequentially obtained through the T times of denoising processing by using data of a sample protein as the constraint condition; D_{KL} represents a function symbol of a relative entropy function; q(G^{t-1}|G^{t}, G⁰, p_{ctx}) represents the small molecule data after the (t−1)^{th} time of noise processing obtained in the process of performing noise processing on the small molecule data after the 0^{th} time of noise processing to obtain the small molecule data after the t^{th} time of noise processing by using the data of the sample protein as the constraint condition; and p_{θ}(G^{t-1}|G^{t}, p_{ctx}) represents the small molecule data after the (t−1)^{th} time of noise processing obtained by performing denoising processing on the small molecule data after the t^{th} time of noise processing by using the data of the sample protein as the constraint condition. By using a heavy parameter skill,

$${G}^{t} = \sqrt{{\overline{α}}_{t}} {G}^{0} + \sqrt{1 - {\overline{α}}_{t}} ϵ$$

may be obtained based on the annotated noise data, where α̅ₜ is a set parameter.

In a possible implementation, the predicted noise data includes predicted type noise data and predicted location noise data, and the annotated noise data includes annotated type noise data and annotated location noise data, where the predicted type noise data is noise data related to types of the sample atoms obtained through prediction, and the predicted location noise data is noise data related to locations of the sample atoms obtained through prediction; and the annotated type noise data is noise data related to the types of the sample atoms obtained through annotation, and the annotated location noise data is noise data related to the locations of the sample atoms obtained through annotation.

Operation 204 includes operation 2041 to operation 2043.

Operation 2041: Determine a first loss based on the predicted type noise data and the annotated type noise data.

In this embodiment of this application, according to a first loss function, the first loss may be determined based on the predicted type noise data and the annotated type noise data. The first loss function is not limited in the embodiments of this application. For example, the first loss function is a relative entropy loss function, a mean absolute error (MAE) loss function, a mean square error (MSE) loss function, or the like. The MAE loss function is also referred to as an L1 loss function, and the MSE loss function is also referred to as an L2 loss function. In one embodiment, the first loss function may alternatively be a loss function obtained by smoothing the L1 loss function by using the L2 loss function, that is, a smoothed L1 loss function.

Operation 2042: Determine a second loss based on the predicted location noise data and the annotated location noise data.

In this embodiment of this application, according to a second loss function, the second loss may be determined based on the predicted location noise data and the annotated location noise data. The second loss function is not limited in the embodiments of this application. For example, the second loss function is a relative entropy loss function, an L1 loss function, an L2 loss function, or an L1 loss function after smoothing.

Operation 2043: Train the neural network model based on the first loss and the second loss, to obtain the noise data determining model.

In this embodiment of this application, operation processing such as weighted summation or weighted averaging may be performed on the first loss and the second loss, to obtain a loss of the neural network model. Training is performed on the neural network model based on the loss of the neural network model, to obtain a trained neural network model. If the trained neural network model meets a training end condition, the trained neural network model is used as the noise data determining model. If the trained neural network model does not meet the training end condition, the trained neural network model is used as next neural network model for training, and next time of training is performed on the neural network model according to operation 201 to operation 204 until the training end condition is met, to obtain the noise data determining model.

Types of atoms are constrained through the first loss of the sample atoms, and locations of the atoms are constrained through the second loss of the sample atoms, thereby improving accuracy of the noise data determining model.

In addition to determining the loss of the neural network model based on the first loss and the second loss, the loss of the neural network model may also be determined based on another loss.

For example, after the predicted noise data is determined, denoising processing may be performed on the sample noisy small molecule data based on the predicted noise data, to obtain denoised small molecule data. The denoised small molecule data includes first data of the plurality of sample atoms. Based on first data of any sample atom and the data of the sample protein, a distance between the sample atom and a surface of the sample protein is determined.

If the distance between the sample atom and the surface of the sample protein is less than a size of the sample atom, a difference obtained by subtracting the distance between the sample atom and the surface of the sample protein from the size of the sample atom is used as a third loss of the sample atom. If the distance between the sample atom and the surface of the sample protein is not less than the size of the sample atom, there is no third loss of the sample atom.

The loss of the neural network model is determined based on the first loss and/or the second loss and/or the third loss of at least one sample atom, and the noise data determining model is obtained through training by using the loss of the neural network model.

Because the distance between the sample atom and the surface of the sample protein is less than the size of the sample atom, there is the third loss of the sample atom, and the third loss of the sample atom is the difference obtained by subtracting the distance between the sample atom and the surface of the sample protein from the size of the sample atom. In this way, locations of atoms in a small molecule are constrained by the third loss of the sample atom, thereby avoiding overlapping of the atoms in the small molecule with a protein, and improving the accuracy of the noise data determining model.

Information (including but not limited to user device information, user personal information, and the like), data (including but not limited to data for analysis, stored data, displayed data, and the like) and signals involved in this application are authorized by a user or fully authorized by all parties, and collection, use, and processing of relevant data need to comply with relevant laws, regulations, and standards of relevant countries and regions. For example, the sample noisy small molecule data and the annotated noise data involved in this application are all obtained under full authorization.

In the foregoing method, a sample graph structure is determined based on data of a plurality of sample atoms in sample noisy small molecule data, prediction is performed on the sample graph structure by using a neural network model, to determine predicted noise data, and training is performed based on the predicted noise data and annotated noise data, to obtain a noise data determining model. Final noise data in to-be-processed noisy small molecule data may be determined by using the noise data determining model, so that denoising processing can be performed on the to-be-processed noisy small molecule data based on the final noise data, to obtain denoised small molecule data. In this way, drug research and development can be performed based on the denoised small molecule data, thereby improving drug research and development efficiency.

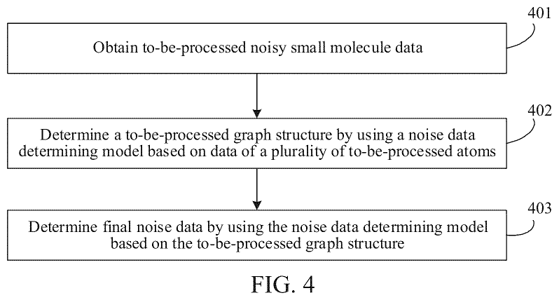

An embodiment of this application further provides a method for determining noise data. The method may be applied to the foregoing implementation environment. Final noise data in to-be-processed noisy small molecule data may be determined by using a noise data determining model. A flowchart of a method for determining noise data according to an embodiment of this application shown in FIG. 4 is used as an example. For case of description, the terminal device 101 or the server 102 performing the method for determining noise data in this embodiment of this application is referred to as an electronic device. The method may be performed by the electronic device. As shown in FIG. 4, the method includes the following operations.

Operation 401: Obtain to-be-processed noisy small molecule data.

The to-be-processed noisy small molecule data is small molecule data with noise data and the to-be-processed noisy small molecule data includes data of a plurality of to-be-processed atoms. Actions “sample noisy small molecule data” used to perform operation 201, as previously described, may be similarly used to perform operation 401. Details are not described herein again.

Operation 402: Determine a to-be-processed graph structure by using a noise data determining model based on the data of the plurality of to-be-processed atoms.

The to-be-processed graph structure includes a plurality of nodes and a plurality of edges, any node represents one to-be-processed atom, any edge represents a distance between to-be-processed atoms corresponding to two nodes at two ends of the edge, and the noise data determining model is obtained through training according to the method for training a noise data determining model in FIG. 2. Actions used to perform operation 202, as previously described, may be similarly used to perform operation 402. Details are not described herein again.

In a possible implementation, operation 402 includes: performing feature extraction on the data of the plurality of to-be-processed atoms by using the noise data determining model, to obtain initial atomic features of the to-be-processed atoms; obtaining data of a to-be-processed protein, and performing feature extraction on the data of the to-be-processed protein by using the noise data determining model, to obtain a feature of the to-be-processed protein; and determining the to-be-processed graph structure by using the noise data determining model based on the initial atomic features of the to-be-processed atoms and the feature of the to-be-processed protein. Actions used to perform Implementation A1, as previously described, may be similarly used to perform operation 402. Details are not described herein again.

In another possible implementation, the to-be-processed noisy small molecule data is initial noise data or is obtained by performing at least once of denoising processing on the initial noise data; and operation 402 includes: obtaining number-of-times-of-denoising information, the number-of-times-of-denoising information representing a number of times of denoising processing required to change the initial noise data to the to-be-processed noisy small molecule data; and determining the to-be-processed graph structure by using the noise data determining model based on the number-of-times-of-denoising information and the data of the plurality of to-be-processed atoms. Actions used to perform Implementation A3 may be similarly used to perform this other possible implementation of operation 402. Details are not described herein again.

Operation 403: Determine final noise data by using the noise data determining model based on the to-be-processed graph structure.

The final noise data is noise data in the to-be-processed noisy small molecule data. Actions used to perform operation 203, as previously described, may be similarly used to perform operation 403. Details are not described herein again.

In a possible implementation, after operation 403, the method further includes: performing denoising processing on the to-be-processed noisy small molecule data based on the final noise data, to obtain first small molecule data; and using the first small molecule data as target small molecule data in response to that the first small molecule data meets a data condition.

In this embodiment of this application, the final noise data may be removed from the to-be-processed noisy small molecule data, to obtain the first small molecule data by performing denoising processing on the to-be-processed noisy small molecule data, and when the first small molecule data meets the data condition, the first small molecule data is used as the target small molecule data.

The first small molecule data meeting the data condition is not limited in the embodiments of this application. For example, the first small molecule data is obtained by performing at least once of denoising processing on the initial noise data. Therefore, when a number of times of denoising processing corresponding to the first small molecule data reaches a set number, the first small molecule data meets the data condition.

For example, if the first small molecule data is obtained by performing t times of denoising processing on the initial noise data, the number of times of denoising processing corresponding to the first small molecule data is t. If t=T, the first small molecule data meets the data condition. If t<T, the first small molecule data does not meet the data condition.

Alternatively, an error between the target small molecule and the first small molecule may be determined based on the to-be-processed noisy small molecule data and the first small molecule data. If the error is in a set range, it is determined that the first small molecule data meets the data condition. If the error is out of the set range, it is determined that the first small molecule data does not meet the data condition.

In a possible implementation, after the performing denoising processing on the to-be-processed noisy small molecule data based on the final noise data, to obtain first small molecule data, the method further includes: determining, in response to that the first small molecule data does not meet the data condition, a reference graph structure by using the noise data determining model based on the first small molecule data; determining reference noise data by using the noise data determining model based on the reference graph structure; performing denoising processing on the first small molecule data based on the reference noise data, to obtain second small molecule data; and using the second small molecule data as the target small molecule data in response to that the second small molecule data meets the data condition.

When the first small molecule data does not meet the data condition, the first small molecule data may be considered as to-be-processed noisy small molecule data. According to operation 402, the reference graph structure is determined by using the noise data determining model based on the first small molecule data. The reference graph structure may be considered as a to-be-processed graph structure. According to operation 403, the reference noise data is determined by using the noise data determining model based on the reference graph structure, where the reference noise data may be considered as final noise data. Therefore, actions used to determine the reference noise data based on the first small molecule data is similar to those described for operation 401 to operation 403. Details are not described herein again.

Next, the reference noise data is removed from the first small molecule data, to obtain the second small molecule data by performing denoising processing on the first small molecule data, and when the second small molecule data meets the data condition, the second small molecule data is used as the target small molecule data. When the second small molecule data does not meet the data condition, the second small molecule data may be used as first small molecule data, reference noise data in the first small molecule data is determined, and the reference noise data is removed from the first small molecule data until the data condition is met, to obtain the target small molecule data.

Information (including but not limited to user device information, user personal information, and the like), data (including but not limited to data for analysis, stored data, displayed data, and the like) and signals involved in this application are authorized by a user or fully authorized by all parties, and collection, use, and processing of relevant data need to comply with relevant laws, regulations, and standards of relevant countries and regions. For example, the to-be-processed noisy small molecule data and the like involved in this application are all obtained under full authorization.

In the foregoing method, a to-be-processed graph structure is determined based on to-be-processed noisy small molecule data, and final noise data is determined by using a noise data determining model based on the to-be-processed graph structure, so that denoising processing can be performed on the to-be-processed noisy small molecule data based on the final noise data, to obtain denoised small molecule data. In this way, drug research and development can be performed based on the denoised small molecule data, thereby improving drug research and development efficiency.

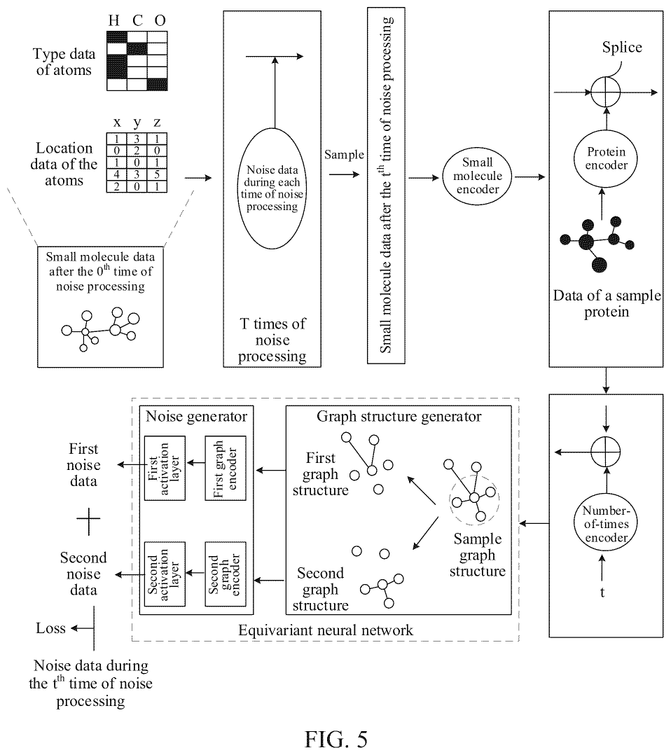

The foregoing describes the method for training a noise data determining model and the method for determining noise data from a perspective of method operations. The following systematically describes a process of training the noise data determining model. FIG. 5 is a schematic diagram of a process of training a noise data determining model according to an embodiment of this application. In this embodiment of this application, the noise data determining model is obtained by performing at least once of training on a neural network model. The neural network model includes a small molecule encoder, a protein encoder, a number-of-times encoder, and an equivariant neural network, where the equivariant neural network includes a graph structure generator and a noise generator. In a process of training the neural network model, only a model parameter is changed, and a model structure remains unchanged. Therefore, the noise data determining model also includes the foregoing network blocks.

First, sample small molecule data is obtained, where the sample small molecule data is denoted as small molecule data after a 0th time of noise processing. The small molecule data after the 0^{th} time of noise processing includes type data of a plurality of atoms and location data of the plurality of atoms, where the type data of the plurality of atoms may be represented as A, and the location data of the plurality of atoms may be represented as R. When an atom is a hydrogen atom, type data of the atom is an element symbol H; when an atom is a carbon atom, type data of the atom is an element symbol C; and when an atom is an oxygen atom, type data of the atom is an element symbol O. In FIG. 5, the small molecule data after the 0^{th} time of noise processing includes type data of five atoms, and the type data of the five atoms is H, C, H, H, and O sequentially. Location data of one atom includes an abscissa (represented as x), an ordinate (represented as y), and a vertical coordinate (represented as z), and may be abbreviated as [x, y, z]. In FIG. 5, the small molecule data after the 0^{th} time of noise processing includes location data of five atoms, and the location data of the five atoms is [1, 3, 1], [0, 2, 0], [1, 0, 1], [4, 3, 5], and [2, 0, 1] sequentially.

Based on noise data during a first time of noise processing, the first time of noise processing may be performed on the small molecule data after the 0^{th} time of noise processing, to obtain small molecule data after the first time of noise processing; and based on noise data during a second time of noise processing, the second time of noise processing is performed on the small molecule data after the first time of noise processing, to obtain small molecule data after the second time of noise processing. The rest is deduced by analogy. In other words, based on noise data during each time of noise processing, T times of noise processing are performed on small molecule data after the 0^{th} time of noise processing, so that small molecule data after the first time of noise processing to a T^{th} time of noise processing may be obtained, where T is a positive integer. One piece of small molecule data is randomly sampled from the small molecule data after the first time of noise processing to the T^{th} time of noise processing, to obtain small molecule data after a t^{th} time of noise processing, where t is a positive integer greater than or equal to one and less than or equal to T.

The small molecule data after the t^{th} time of noise processing is the sample noisy small molecule data described above. Because t times of noise processing are performed on the small molecule data after the 0^{th} time of noise processing, type data of atoms included in the small molecule data after the t^{th} time of noise processing carries specific noise data, and the type data of the atoms carrying the specific noise data may be denoted as type data of sample atoms. Similarly, location data of the atoms included in the small molecule data after the t^{th} time of noise processing also carries specific noise data, and the location data of the atoms carrying the specific noise data is denoted as location data of the sample atoms.

The small molecule data after the t^{th} time of noise processing is inputted into the small molecule encoder, the small molecule encoder performs encoding processing on the type data of the sample atoms, to obtain type features of the sample atoms, and the small molecule encoder performs encoding processing on the location data of the sample atoms, to obtain location features of the sample atoms. An initial atomic feature of any sample atom includes the type feature of the sample atom and the location feature of the sample atom. The type features of the plurality of sample atoms may be represented as At, and the location features of the plurality of sample atoms may be represented as Rt.

Data of a sample protein may be inputted into a protein encoder, and the protein encoder performs feature extraction on the data of the sample protein, to obtain a feature of the sample protein, where the feature of the sample protein may be represented as Cp. The feature of the sample protein, the type features of the plurality of sample atoms, and the location features of the plurality of sample atoms are spliced to obtain a first spliced feature, where the first spliced feature may be represented by [At, Cp], Rt. The first spliced feature includes the first atomic features of the sample atoms described above.

Sample number-of-times-of-noise-processing information may also be obtained, that is, t may be obtained. t is inputted into the number-of-times encoder, and the number-of-times encoder performs encoding processing on t, to obtain a sample number-of-times-of-noise-processing feature, where the sample number-of-times-of-noise-processing feature may be represented as te. The first spliced feature and the sample number-of-times-of-noise-processing feature are spliced to obtain a second spliced feature, where the second spliced feature may be represented as [At, Cp, te], Rt. The second spliced feature includes the fifth atomic features of the sample atoms described above.

Next, the second spliced feature is inputted into the equivariant neural network, and the graph structure generator included in the equivariant neural network constructs a sample graph structure based on the second spliced feature. The sample graph structure includes a plurality of sample nodes and a plurality of sample edges, any sample node is a fifth atomic feature of one sample atom, and any sample edge is a fifth distance between two sample atoms at two ends of the edge determined based on fifth atomic features of the two sample atoms. The graph structure generator deletes sample edges with fifth distances not greater than a reference distance from the sample graph structure, to obtain a first graph structure; and the graph structure generator deletes sample edges with fifth distances greater than the reference distance from the sample graph structure, to obtain a second graph structure. A dashed circle shown in FIG. 5 represents a range region in which sample atoms with fifth distances from a sample atom located at a center of the circle not greater than the reference distance are located.

The first graph structure is inputted into a first graph encoder, to obtain a first graph feature, where the first graph feature includes the sixth atomic features of the sample atoms described above; and the first graph feature is inputted into a first activation layer, to obtain first noise data, where the first noise data includes first type noise data and first location noise data. Similarly, the second graph structure is inputted into a second graph encoder, to obtain a second graph feature, where the second graph feature includes the seventh atomic features of the sample atoms described above; and the second graph feature is inputted into a second activation layer, to obtain second noise data, where the second noise data includes second type noise data and second location noise data.

Then, noise data during the t^{th} time of noise processing is obtained, where the noise data during the t^{th} time of noise processing is used as annotated noise data. The first noise data and the second noise data are used as predicted noise data. A loss of the neural network model may be determined by using the predicted noise data and the annotated noise data, to perform training on the neural network model once based on the loss of the neural network model, to obtain a trained neural network model. When the trained neural network model meets a training end condition, the trained neural network model is the noise data determining model. When the trained neural network model does not meet the training end condition, the trained neural network model is next neural network model for training, and next time of training is performed on the neural network model in the manner shown in FIG. 5 until the training end condition is met, to obtain the noise data determining model.

In this embodiment of this application, randomly generated Gaussian noise data may be obtained. The Gaussian noise data is used as the small molecule data processed after the T^{th} time of noise processing. The noise data determining model determines noise data in the small molecule data after the T^{th} time of noise processing, where the noise data may be denoted as noise data during the T^{th} time of noise processing, and performs denoising processing on the small molecule data after the T^{th} time of noise processing based on the noise data during the T^{th} time of noise processing, to obtain small molecule data after a (t−1)^{th}time of noise processing; and the noise data determining model determines noise data in the small molecule data after the (t−1)^{th} time of noise processing, where the noise data may be denoted as noise data during the (t−1)^{th} time of noise processing, and performs denoising processing on the small molecule data after the (t−1)^{th} time of noise processing based on the noise data during the (t−1)^{th} time of noise processing, to obtain small molecule data after a (T-2)^{th} time of noise processing. The rest is deduced by analogy. In other words, based on noise data during each time of noise processing, T times of denoising processing are performed on the small molecule data after the T^{th} time of noise processing, so that the small molecule data after the 0^{th} time of noise processing, namely, the target small molecule data described above, may be obtained.

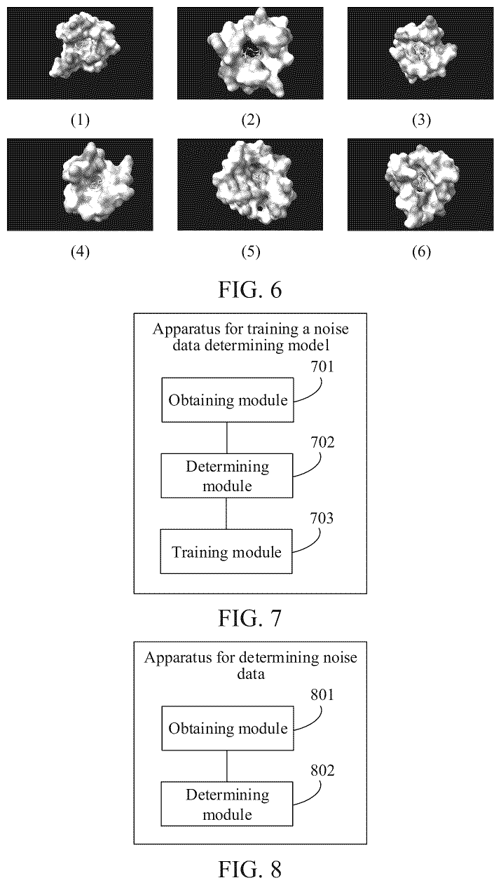

The target small molecule data is data describing a target small molecule. FIG. 6 is a schematic diagram of a target small molecule according to an embodiment of this application. (1) to (6) in FIG. 6 show six target small molecules respectively.

For a process of determining noise data in small molecule data after any time of noise processing by using the noise data determining model, refer to the process of determining the predicted noise data based on the small molecule data after the t^{th} time of noise processing in FIG. 5, where the predicted noise data includes the first noise data and the second noise data. Implementation principles of the two are similar, and details are not described herein again.

In this embodiment of this application, the noise data determining model performs the T times of denoising processing on the Gaussian noise data, to implement a process of restoring, based on a thermal diffusion theory, atoms in the small molecule data constantly approaching a stable state from an unstable state. Through the data of the sample protein, data of the target small molecule that can bind to the sample protein is generated, so that the drug research and development rate can be sped up. The noise data in the small molecule data after any time of noise processing is determined, and denoising processing is performed on small molecule data after the noise processing based on the noise data, so that denoised small molecule data can be generated at a time, in other words, type data of atoms and location data of the atoms included in the small molecule data are generated at a time. In this way, the generation rate is increased, and the generation time is shortened, and the generation at a time can avoid an accumulative error. In addition, Gaussian noise is randomly generated, so that a number of atoms included in the small molecule can be customized.

In the related art, another neural network model may be used to generate the target small molecule data. A model that can generate the target small molecule data in the related art may be denoted as a small molecule generation model. In this embodiment of this application, a small molecule generation model 1, a small molecule generation model 2, and the noise data determining model are obtained through training by using a same dataset, and performance of the three models in generating the target small molecule data is tested. Model performance may be evaluated by using a score indicator 1 to a score indicator 6, and obtained results are shown in Table 1 below.

TABLE 1

| | Small molecule | Small molecule | Noise data |
|---|---|---|---|
| | generation | generation | determining |
| | model 1 | model 2 | model |
| Score indicator 1 (↓) | −6.144 | −6.215 | −6.627 |
| Score indicator 2 (↑) | 0.238 | 0.267 | 0.4258 |
| Score indicator 3 (↑) | 0.369 | 0.502 | 0.51 |
| Score indicator 4 (↑) | 0.590 | 0.675 | 0.563 |
| Score indicator 5 (↑) | −0.140 | 0.257 | 3.451 |
| Score indicator 6 (↑) | 4.027 | 4.787 | 4.797 |

The symbol “↓” represents that a smaller value of a score indicator indicates better model performance. The symbol “↑” represents that a larger value of a score indicator indicates better model performance. As shown in Table 1, the performance of the noise data determining model is superior to the small molecule generation model 1 and the small molecule generation model 2.

FIG. 7 is a structural schematic diagram of an apparatus for training a noise data determining model according to an embodiment of this application. As shown in FIG. 7, the apparatus includes:
an obtaining module 701, configured to obtain sample noisy small molecule data and annotated noise data, the sample noisy small molecule data being small molecule data with noise data and the sample noisy small molecule data including data of a plurality of sample atoms, and the annotated noise data being noise data obtained from the sample noisy small molecule data through annotation;
a determining module 702, configured to output a sample graph structure by using a neural network model based on the data of the plurality of sample atoms, the sample graph structure including a plurality of sample nodes and a plurality of sample edges, any sample node representing data of one sample atom, and any sample edge representing a distance between sample atoms corresponding to two sample nodes at two ends of the sample edge,
the determining module 702 being further configured to perform prediction on the sample graph structure by using the neural network model, to obtain predicted noise data, the predicted noise data being noise data obtained from the sample noisy small molecule data through prediction; and
a training module 703, configured to train the neural network model based on the predicted noise data and the annotated noise data, to obtain a noise data determining model, the noise data determining model being configured to determine final noise data in to-be-processed noisy small molecule data.

In a possible implementation, the determining module 702 is configured to perform feature extraction on the data of the plurality of sample atoms respectively by using the neural network model, to obtain initial atomic features of the sample atoms; obtain data of a sample protein, and performing feature extraction on the data of the sample protein by using the neural network model, to obtain a feature of the sample protein; and determine the sample graph structure by using the neural network model based on the initial atomic features of the sample atoms and the feature of the sample protein.

In a possible implementation, the determining module 702 is configured to fuse, for any sample atom, the initial atomic feature of the any sample atom and the feature of the sample protein by using the neural network model, to obtain a first atomic feature of the any sample atom; determine a first distance between each two sample atoms based on first atomic features of the sample atoms; and determine the sample graph structure based on the first atomic features of the sample atoms and the first distance between each two sample atoms.

In a possible implementation, the sample noisy small molecule data is initial noise data or is obtained by performing at least once of denoising processing on the initial noise data; and
the determining module 702 is configured to obtain sample number-of-times-of-denoising information, the sample number-of-times-of-denoising information representing a number of times of denoising processing performed to change the initial noise data to the sample noisy small molecule data; and determine the sample graph structure by using the neural network model based on the sample number-of-times-of-denoising information and the data of the plurality of sample atoms.

In a possible implementation, the determining module 702 is configured to perform feature extraction on the sample number-of-times-of-denoising information by using the neural network model, to obtain a sample number-of-times-of-denoising feature; perform feature extraction on the data of the plurality of sample atoms respectively by using the neural network model, to obtain initial atomic features of the sample atoms; and determine the sample graph structure by using the neural network model based on the sample number-of-times-of-denoising feature and the initial atomic features of the sample atoms.

In a possible implementation, the determining module 702 is configured to fuse, for any sample atom, the initial atomic feature of the any sample atom and the sample number-of-times-of-denoising feature by using the neural network model, to obtain a second atomic feature of the any sample atom; determine a second distance between each two sample atoms based on second atomic features of the sample atoms; and determine the sample graph structure based on the second atomic features of the sample atoms and the second distance between each two sample atoms.

In a possible implementation, the determining module 702 is configured to fuse, for any sample atom, the initial atomic feature of the any sample atom, the sample number-of-times-of-denoising feature, and a feature of a sample protein by using the neural network model, to obtain a third atomic feature of the any sample atom; determine a third distance between each two sample atoms based on third atomic features of the sample atoms; and determine the sample graph structure based on the third atomic features of the sample atoms and the third distance between each two sample atoms.

In a possible implementation, the determining module 702 is configured to perform feature extraction on the sample graph structure by using the neural network model, to obtain to-be-processed atomic features of the sample atoms; determine, based on the to-be-processed atomic features of the sample atoms, at least one of predicted type noise data or predicted location noise data by using the neural network model, the predicted type noise data being noise data related to types of the sample atoms obtained through prediction, and the predicted location noise data being noise data related to locations of the sample atoms obtained through prediction; and use the at least one of the predicted type noise data or the predicted location noise data as the predicted noise data.

In a possible implementation, the determining module 702 is configured to delete, by using the neural network model, a first edge from the plurality of sample edges included in the sample graph structure, to obtain a first graph structure, a distance represented by the first edge being not greater than a reference distance; determine first noise data by using the neural network model based on the first graph structure; and determine the predicted noise data based on the first noise data.

In a possible implementation, the determining module 702 is configured to delete, by using the neural network model, a second edge from the plurality of sample edges included in the sample graph structure, to obtain a second graph structure, a distance represented by the second edge being greater than a reference distance; determine second noise data by using the neural network model based on the second graph structure; and determine the predicted noise data based on the second noise data.

In a possible implementation, the predicted noise data includes predicted type noise data and predicted location noise data, and the annotated noise data includes annotated type noise data and annotated location noise data; and
the training module 703 is configured to determine a first loss based on the predicted type noise data and the annotated type noise data; determine a second loss based on the predicted location noise data and the annotated location noise data; and train the neural network model based on the first loss and the second loss, to obtain the noise data determining model.

In the foregoing apparatus, a sample graph structure is determined based on data of a plurality of sample atoms in sample noisy small molecule data, prediction is performed on the sample graph structure by using a neural network model, to determine predicted noise data, and training is performed based on the predicted noise data and annotated noise data, to obtain a noise data determining model. Final noise data in to-be-processed noisy small molecule data may be determined by using the noise data determining model, so that denoising processing can be performed on the to-be-processed noisy small molecule data based on the final noise data, to obtain denoised small molecule data. In this way, drug research and development can be performed based on the denoised small molecule data, thereby improving drug research and development efficiency.

When the apparatus provided in FIG. 7 implements functions of the apparatus, division of the foregoing functional modules is merely used as an example for description. In actual application, the foregoing functions may be allocated to different functional modules for implementation according to a requirement, in other words, an internal structure of a device is divided into different functional modules, to implement all or some of the functions described above. In addition, the apparatus provided in the foregoing embodiments and the method embodiments fall within a same conception. That is, the apparatus in FIG. 7 is configured to perform the previously described method embodiments. Details are not described herein again.

FIG. 8 is a structural schematic diagram of an apparatus for determining noise data according to an embodiment of this application. As shown in FIG. 8, the apparatus includes:
an obtaining module 801, configured to obtain to-be-processed noisy small molecule data, the to-be-processed noisy small molecule data being small molecule data with noise data and the to-be-processed noisy small molecule data including data of a plurality of to-be-processed atoms; and
a determining module 802, configured to determine a to-be-processed graph structure by using a noise data determining model based on the data of the plurality of to-be-processed atoms, the to-be-processed graph structure including a plurality of nodes and a plurality of edges, any node representing one to-be-processed atom, any edge representing a distance between to-be-processed atoms corresponding to two nodes at two ends of the edge, and the noise data determining model being obtained through training according to the method for training a noise data determining model according to any one of descriptions in the first aspect,
the determining module 802 being further configured to determine final noise data by using the noise data determining model based on the to-be-processed graph structure, the final noise data being noise data in the to-be-processed noisy small molecule data.

In a possible implementation, the determining module 802 is configured to perform feature extraction on the data of the plurality of to-be-processed atoms by using the noise data determining model, to obtain initial atomic features of the to-be-processed atoms; obtain data of a to-be-processed protein, and perform feature extraction on the data of the to-be-processed protein by using the noise data determining model, to obtain a feature of the to-be-processed protein; and determine the to-be-processed graph structure by using the noise data determining model based on the initial atomic features of the to-be-processed atoms and the feature of the to-be-processed protein.

In a possible implementation, the to-be-processed noisy small molecule data is initial noise data or is obtained by performing at least once of denoising processing on the initial noise data; and
the determining module 802 is configured to obtain number-of-times-of-denoising information, the number-of-times-of-denoising information representing a number of times of denoising processing performed to change the initial noise data to the to-be-processed noisy small molecule data; and determine the to-be-processed graph structure by using the noise data determining model based on the number-of-times-of-denoising information and the data of the plurality of to-be-processed atoms.

In a possible implementation, the apparatus further includes:
a denoising module, configured to perform denoising processing on the to-be-processed noisy small molecule data based on the final noise data, to obtain first small molecule data; and
the determining module 802 is further configured to use the first small molecule data as target small molecule data in response to that the first small molecule data meets a data condition.

In a possible implementation, the apparatus further includes:
the determining module 802, further configured to determine, in response to that the first small molecule data does not meet the data condition, a reference graph structure by using the noise data determining model based on the first small molecule data; and determine reference noise data by using the noise data determining model based on the reference graph structure;
the denoising module, further configured to perform denoising processing on the first small molecule data based on the reference noise data, to obtain second small molecule data; and
the determining module 802, further configured to use the second small molecule data as the target small molecule data in response to that the second small molecule data meets the data condition.

In the foregoing apparatus, a to-be-processed graph structure is determined based on to-be-processed noisy small molecule data, and final noise data is determined by using a noise data determining model based on the to-be-processed graph structure, so that denoising processing can be performed on the to-be-processed noisy small molecule data based on the final noise data, to obtain denoised small molecule data. In this way, drug research and development can be performed based on the denoised small molecule data, thereby improving drug research and development efficiency.

When the apparatus provided in FIG. 8 implements functions of the apparatus, division of the foregoing functional modules is merely used as an example for description. In actual application, the foregoing functions may be allocated to different functional modules for implementation according to a requirement, in other words, an internal structure of a device is divided into different functional modules, to implement all or some of the functions described above. In addition, the apparatus provided in the foregoing embodiments and the method embodiments fall within a same conception. That is, the apparatus of FIG. 8 is configured to perform the previously described method embodiments. Details are not described herein again.

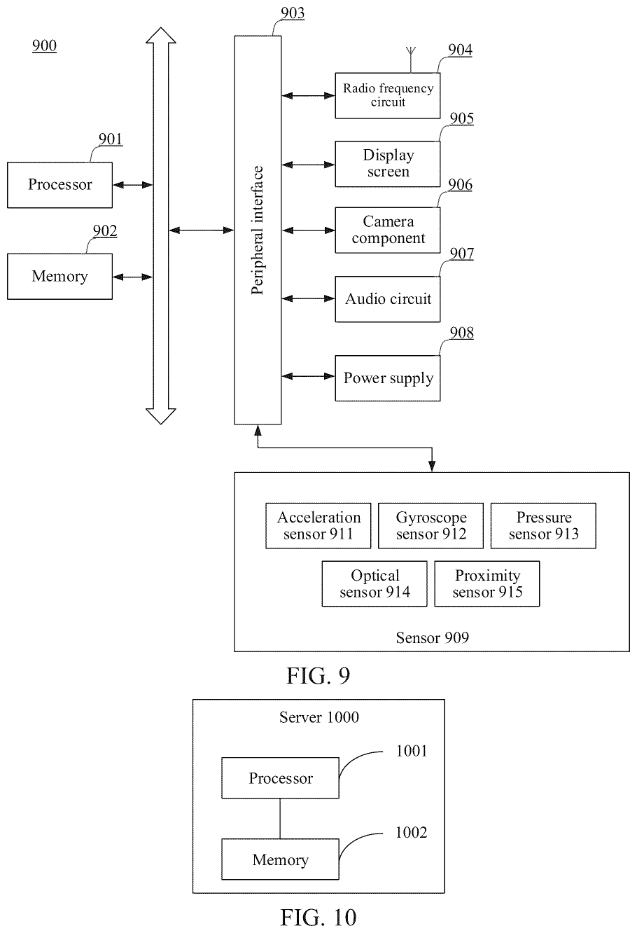

FIG. 9 is a structural block diagram of a terminal device 900 according to an exemplary embodiment of this application. The terminal device 900 includes a processor 901 and a memory 902.

The processor 901 may include one or more processing cores, for example, a 4-core processor or an 8-core processor. The processor 901 may be implemented by using at least one hardware form of a digital signal processor (DSP), a field-programmable gate array (FPGA), or a programmable logic array (PLA). The processor 901 further includes a main processor and a coprocessor. The main processor is configured to process data in an active state, also referred to as a central processing unit (CPU). The coprocessor is a low-power consumption processor configured to process data in a standby state. In some embodiments, the processor 901 may be integrated with a graphics processing unit (GPU). The GPU is configured to render and draw content that needs to be displayed on a display. In some embodiments, the processor 901 may further include an artificial intelligence (AI) processor. The AI processor is configured to process computing operations related to machine learning.

The memory 902 may include one or more computer-readable storage media. The computer-readable storage media may be non-transient. The memory 902 may also include a high-speed random access memory, as well as non-volatile memory, such as one or more disk storage devices and flash storage devices. In some embodiments, a non-transitory computer-readable storage medium in the memory 902 is configured to store at least one computer program, the at least one computer program being configured to be executed by the processor 901 to implement the method for training a noise data determining model or the method for determining noise data provided in the method embodiments of this application.

In some embodiments, the terminal device 900 may include a peripheral interface 903 and at least one peripheral. The peripheral includes: at least one of a radio frequency circuit 904, a display screen 905, a camera component 906, an audio circuit 907, or a power supply 908.

In some embodiments, the terminal device 900 may further include one or more sensors 909. The one or more sensors 909 include, but are not limited to: an acceleration sensor 911, a gyroscope sensor 912, a pressure sensor 913, an optical sensor 914, and a proximity sensor 915.

A person skilled in the art may understand that the structure shown in FIG. 9 does not constitute a limitation to the terminal device 900, and the terminal may include more components or fewer components than those shown in the figure, or some components may be combined, or a different component deployment may be used.

FIG. 10 is a schematic structural diagram of a server according to an embodiment of this application. A server 1000 may vary greatly due to different configurations or performance, and may include one or more processors 1001 and one or more memories 1002. The one or more memories 1002 have at least one computer program stored therein, the at least one computer program being loaded and executed by the one or more processors 1001 to implement the method for training a noise data determining model or the method for determining noise data provided in the foregoing method embodiments. For example, the processors 1001 is a CPU. Certainly, the server 1000 may also have a wired or wireless network interface, a keyboard, an input/output interface and other components to facilitate input/output. The server 1000 may also include other components for implementing device functions. Details are not described herein.

In an exemplary embodiment, a non-transitory computer-readable storage medium is further provided, the storage medium having at least one computer program stored therein, the at least one computer program being loaded and executed by a processor, to cause an electronic device to implement the method for training a noise data determining model or the method for determining noise data according to any one of the foregoing descriptions.

In one embodiment, the computer-readable storage medium may be a read-only memory (ROM), a random access memory (RAM), a compact disc ROM (CD-ROM), a magnetic tape, a floppy disk, an optical data storage device, or the like.

In an exemplary embodiment, a computer program product is further provided, the computer program product having at least one computer program stored therein, the at least one computer program being loaded and executed by a processor, to cause an electronic device to implement the method for training a noise data determining model or the method for determining noise data according to any one of the foregoing descriptions.

In addition, the term “plurality of” mentioned in this specification means two or more. The term “and/or” describes an association relationship for describing associated objects and represents that three relationships may exist. For example, A and/or B may represent the following three cases: Only A exists, both A and B exist, and only B exists. The character “/” in this specification generally represents an “or” relationship between the associated objects.

The sequence numbers of the foregoing embodiments of this application are merely for description purpose, and are not intended to indicate priorities of the embodiments.

The foregoing descriptions are merely examples of the embodiments of this application, but are not intended to limit this application. Any modification, equivalent replacement, or improvement made without departing from the principle of this application shall fall within the protection scope of this application.

What is claimed is:

1. A method for training a noise data determining model, performed by an electronic device, the method comprising:
obtaining sample noisy small molecule data and annotated noise data, the sample noisy small molecule data being small molecule data with noise data and comprising data of a plurality of sample atoms, and the annotated noise data being noise data obtained from the sample noisy small molecule data through annotation;
outputting a sample graph structure by using a neural network model based on the data of the plurality of sample atoms, the sample graph structure comprising a plurality of sample nodes and a plurality of sample edges, each sample node representing data of a respective one of the plurality of sample atoms, and each sample edge representing a distance between respective sample atoms corresponding to two sample nodes at two ends of the sample edge;
performing prediction on the sample graph structure by using the neural network model, to obtain predicted noise data, the predicted noise data being noise data obtained from the sample noisy small molecule data through prediction; and
training the neural network model based on the predicted noise data and the annotated noise data, to obtain a noise data determining model, the noise data determining model being configured to determine final noise data in to-be-processed noisy small molecule data.

2. The method according to claim 1, wherein the outputting a sample graph structure by using a neural network model based on the data of the plurality of sample atoms comprises:
performing feature extraction on the data of the plurality of sample atoms respectively by using the neural network model, to obtain initial atomic features of the sample atoms;
obtaining data of a sample protein, and performing feature extraction on the data of the sample protein by using the neural network model, to obtain a feature of the sample protein; and
determining the sample graph structure by using the neural network model based on the initial atomic features of the sample atoms and the feature of the sample protein.

3. The method according to claim 2, wherein the determining the sample graph structure by using the neural network model based on the initial atomic features of the sample atoms and the feature of the sample protein comprises:
fusing, for each sample atom, the initial atomic feature of the each sample atom and the feature of the sample protein by using the neural network model, to obtain a first atomic feature of the each sample atom;
determining a first distance between each two sample atoms based on first atomic features of the sample atoms; and
determining the sample graph structure based on the first atomic features of the sample atoms and the first distance between each two sample atoms.

4. The method according to claim 1, wherein the sample noisy small molecule data is initial noise data or is obtained by performing denoising processing on the initial noise data at least once; and
the outputting a sample graph structure by using a neural network model based on the data of the plurality of sample atoms comprises:
obtaining sample number-of-times-of-denoising information, the sample number-of-times-of-denoising information representing a number of times of denoising processing performed to change the initial noise data to the sample noisy small molecule data; and
determining the sample graph structure by using the neural network model based on the sample number-of-times-of-denoising information and the data of the plurality of sample atoms.

5. The method according to claim 4, wherein the determining the sample graph structure by using the neural network model based on the sample number-of-times-of-denoising information and the data of the plurality of sample atoms comprises:
performing feature extraction on the sample number-of-times-of-denoising information by using the neural network model, to obtain a sample number-of-times-of-denoising feature;
performing feature extraction on the data of the plurality of sample atoms respectively by using the neural network model, to obtain initial atomic features of the sample atoms; and
determining the sample graph structure by using the neural network model based on the sample number-of-times-of-denoising feature and the initial atomic features of the sample atoms.

6. The method according to claim 5, wherein the determining the sample graph structure by using the neural network model based on the sample number-of-times-of-denoising feature and the initial atomic features of the sample atoms comprises:
fusing, for each sample atom, the initial atomic feature of the each sample atom and the sample number-of-times-of-denoising feature by using the neural network model, to obtain a second atomic feature of the each sample atom;
determining a second distance between each two sample atoms based on second atomic features of the sample atoms; and
determining the sample graph structure based on the second atomic features of the sample atoms and the second distance between each two sample atoms.

7. The method according to claim 5, wherein the determining the sample graph structure by using the neural network model based on the sample number-of-times-of-denoising feature and the initial atomic features of the sample atoms comprises:
fusing, for each sample atom, the initial atomic feature of the each sample atom, the sample number-of-times-of-denoising feature, and a feature of a sample protein by using the neural network model, to obtain a third atomic feature of the each sample atom;
determining a third distance between each two sample atoms based on third atomic features of the sample atoms; and
determining the sample graph structure based on the third atomic features of the sample atoms and the third distance between each two sample atoms.

8. The method according to claim 1, wherein the performing prediction on the sample graph structure by using the neural network model, to obtain predicted noise data comprises:
performing feature extraction on the sample graph structure by using the neural network model, to obtain to-be-processed atomic features of the sample atoms;
determining, based on the to-be-processed atomic features of the sample atoms, at least one of predicted type noise data or predicted location noise data by using the neural network model, the predicted type noise data being noise data related to types of the sample atoms obtained through prediction, and the predicted location noise data being noise data related to locations of the sample atoms obtained through prediction; and
using the at least one of the predicted type noise data or the predicted location noise data as the predicted noise data.

9. The method according to claim 1, wherein the performing prediction on the sample graph structure by using the neural network model, to obtain predicted noise data comprises:
deleting, by using the neural network model, a first edge from the plurality of sample edges comprised in the sample graph structure, to obtain a first graph structure, a distance represented by the first edge being not greater than a reference distance;
determining first noise data by using the neural network model based on the first graph structure; and
determining the predicted noise data based on the first noise data.

10. The method according to claim 1, wherein the performing prediction on the sample graph structure by using the neural network model, to obtain predicted noise data comprises:
deleting, by using the neural network model, a second edge from the plurality of sample edges comprised in the sample graph structure, to obtain a second graph structure, a distance represented by the second edge being greater than a reference distance;
determining second noise data by using the neural network model based on the second graph structure; and
determining the predicted noise data based on the second noise data.

11. The method according to claim 1, wherein the predicted noise data comprises predicted type noise data and predicted location noise data, and the annotated noise data comprises annotated type noise data and annotated location noise data; and
the training the neural network model based on the predicted noise data and the annotated noise data, to obtain a noise data determining model comprises:
determining a first loss based on the predicted type noise data and the annotated type noise data;
determining a second loss based on the predicted location noise data and the annotated location noise data; and
training the neural network model based on the first loss and the second loss, to obtain the noise data determining model.

12. A method for determining noise data, performed by an electronic device, the method comprising:
obtaining to-be-processed noisy small molecule data, the to-be-processed noisy small molecule data being small molecule data with noise data and the to-be-processed noisy small molecule data comprising data of a plurality of to-be-processed atoms;
determining a to-be-processed graph structure by using a noise data determining model based on the data of the plurality of to-be-processed atoms, the to-be-processed graph structure comprising a plurality of nodes and a plurality of edges, each node representing a respective one to-be-processed atom, each edge representing a respective distance between to-be-processed atoms corresponding to two nodes at two ends of the edge; and
determining final noise data by using the noise data determining model based on the to-be-processed graph structure, the final noise data being noise data in the to-be-processed noisy small molecule data.

13. The method according to claim 12, wherein the determining a to-be-processed graph structure by using a noise data determining model based on the data of the plurality of to-be-processed atoms comprises:
performing feature extraction on the data of the plurality of to-be-processed atoms by using the noise data determining model, to obtain initial atomic features of the to-be-processed atoms;
obtaining data of a to-be-processed protein, and performing feature extraction on the data of the to-be-processed protein by using the noise data determining model, to obtain a feature of the to-be-processed protein; and
determining the to-be-processed graph structure by using the noise data determining model based on the initial atomic features of the to-be-processed atoms and the feature of the to-be-processed protein.

14. The method according to claim 12, wherein the to-be-processed noisy small molecule data is initial noise data or is obtained by performing denoising processing on the initial noise data at least once; and
the determining a to-be-processed graph structure by using a noise data determining model based on the data of the plurality of to-be-processed atoms comprises:
obtaining number-of-times-of-denoising information, the number-of-times-of-denoising information representing a number of times of denoising processing performed to change the initial noise data to the to-be-processed noisy small molecule data; and
determining the to-be-processed graph structure by using the noise data determining model based on the number-of-times-of-denoising information and the data of the plurality of to-be-processed atoms.

15. The method according to claim 12, wherein the method further comprises:
performing denoising processing on the to-be-processed noisy small molecule data based on the final noise data, to obtain first small molecule data; and
using the first small molecule data as target small molecule data in response to the first small molecule data meeting a data condition.

16. The method according to claim 15, wherein the method further comprises:
determining, in response to the first small molecule data not meeting the data condition, a reference graph structure by using the noise data determining model based on the first small molecule data;
determining reference noise data by using the noise data determining model based on the reference graph structure;
performing denoising processing on the first small molecule data based on the reference noise data, to obtain second small molecule data; and
using the second small molecule data as the target small molecule data in response to the second small molecule data meeting the data condition.

17. An electronic device comprising:
a memory storing a plurality of instructions; and
a processor configured to execute the plurality of instructions, wherein upon execution of the plurality of instructions, the processor is configured to cause the electric device to:
obtain sample noisy small molecule data and annotated noise data, the sample noisy small molecule data being small molecule data with noise data and comprising data of a plurality of sample atoms, and the annotated noise data being noise data obtained from the sample noisy small molecule data through annotation;
output a sample graph structure by using a neural network model based on the data of the plurality of sample atoms, the sample graph structure comprising a plurality of sample nodes and a plurality of sample edges, each sample node representing data of a respective one of the plurality of sample atoms, and each sample edge representing a distance between respective sample atoms corresponding to two sample nodes at two ends of the sample edge;
perform prediction on the sample graph structure by using the neural network model, to obtain predicted noise data, the predicted noise data being noise data obtained from the sample noisy small molecule data through prediction; and
train the neural network model based on the predicted noise data and the annotated noise data, to obtain a noise data determining model, the noise data determining model being configured to determine final noise data in to-be-processed noisy small molecule data.

18. The electronic device according to claim 17, wherein in order to output the sample graph structure by using the neural network model based on the data of the plurality of sample atoms, the processor, upon execution of the plurality of instructions, is configured to:
perform feature extraction on the data of the plurality of sample atoms respectively by using the neural network model, to obtain initial atomic features of the sample atoms;
obtain data of a sample protein, and perform feature extraction on the data of the sample protein by using the neural network model, to obtain a feature of the sample protein; and
determine the sample graph structure by using the neural network model based on the initial atomic features of the sample atoms and the feature of the sample protein.

19. The electronic device according to claim 17, wherein the sample noisy small molecule data is initial noise data or is obtained by performing denoising processing on the initial noise data at least once; and
in order to output the sample graph structure by using the neural network model based on the data of the plurality of sample atoms, the processor, upon execution of the plurality of instructions, is configured to:
obtain sample number-of-times-of-denoising information, the sample number-of-times-of-denoising information representing a number of times of denoising processing performed to change the initial noise data to the sample noisy small molecule data; and
determining the sample graph structure by using the neural network model based on the sample number-of-times-of-denoising information and the data of the plurality of sample atoms.

20. The electronic device according to claim 17, wherein in order to perform prediction on the sample graph structure by using the neural network model, to obtain predicted noise data, the processor, upon execution of the plurality of instructions, is configured to:
perform feature extraction on the sample graph structure by using the neural network model, to obtain to-be-processed atomic features of the sample atoms;
determine, based on the to-be-processed atomic features of the sample atoms, at least one of predicted type noise data or predicted location noise data by using the neural network model, the predicted type noise data being noise data related to types of the sample atoms obtained through prediction, and the predicted location noise data being noise data related to locations of the sample atoms obtained through prediction; and
use the at least one of the predicted type noise data or the predicted location noise data as the predicted noise data.
